(19) United States
(12) Patent Application Publication
(10) Pub. No.: US 2025/0171558 A1
(43) Pub. Date: May 29, 2025

(54) HUMAN IgG Fc DOMAIN VARIANTS WITH IMPROVED EFFECTOR FUNCTION

(71) Applicant: The Rockefeller University, New York, NY (US)

(72) Inventors: Jeffrey V. Ravetch, New York, NY (US); Stylianos Bournazos, New York, NY (US)

(73) Assignee: The Rockefeller University, New York, NY (US)

(21) Appl. No.: 19/054,403

(22) Filed: Feb. 14, 2025

Related U.S. Application Data
(63) Continuation of application No. 17/699,716, filed on Mar. 21, 2022.
Application No. 17/699,716 is a continuation of application No. 16/424,639, filed on May 29, 2019, now abandoned.
Application No. 16/424,639 is a continuation of application No. PCT/US2018/065103, filed on Dec. 12, 2018.
(60) Provisional application No. 62/607,591, filed on Dec. 19, 2017.

(51) Int. Cl. C07K 16/30; A61K 39/00; C07K 16/00; C07K 16/28; C07K 16/46; C12N 15/85; G01N 33/563

(52) U.S. Cl. CPC C07K 16/30; C07K 16/00; C07K 16/2887; C07K 16/462; C12N 15/85; G01N 33/563; A61K 2039/505; C07K 2317/21; C07K 2317/24; C07K 2317/52; C07K 2317/732; C07K 2317/734; C07K 2317/94; C07K 2319/035; C07K 2319/33; C07K 2319/35; C12N 2015/8518

(57) ABSTRACT

The present invention relates to human IgG Fc domain variants with improved effector function and uses thereof.

### CROSS-REFERENCE TO RELATED APPLICATIONS

This application is a continuation application of U.S. patent application Ser. No. 17/699,716, filed on Mar. 31, 2022, which is a continuation application of U.S. patent application Ser. No. 16/424,639, filed on May 29, 2019, which is now abandoned, which is a continuation application of International Application No. PCT/US2018/065103, filed on Dec. 12, 2018, which is entitled to priority pursuant to 35 U.S.C. § 119(e) to U.S. Provisional Patent Application No. 62/607,591, filed on Dec. 19, 2017. Each disclosure is incorporated herein by reference in its entirety.

### GOVERNMENT INTERESTS

This invention was made with government support under P01 AI100148 awarded by NIAID and NIH. The government has certain rights in the invention.

### REFERENCE TO AN ELECTRONIC SEQUENCE LISTING

The contents of the electronic sequence listing (070413.20838SeqList.xml; Size: 5,760 bytes; and Date of Creation: Feb. 14, 2025) is herein incorporated by reference in its entirety.

### FIELD OF THE INVENTION

This invention relates to human IgG Fc domain variants with improved effector function and uses thereof.

### BACKGROUND OF THE INVENTION

Extensive experience from the clinical use of a number of FDA-approved monoclonal antibodies (mAbs) for the treatment of inflammatory and neoplastic disorders strongly suggests that the therapeutic potential of an antibody is highly dependent on interactions of the IgG Fc domain with its cognate receptors, Fcγ receptors (FcγR) expressed on the surface of effector leukocytes to mediate a range of Fc effector functions (Nimmerjahn et al., Cancer Immun 12, 13 (2012)). For example, the therapeutic outcome of a number of mAbs has been associated with allelic variants of FcγR genes that affect the receptor capacity for IgG binding (Nimmerjahn et al., Cancer Immun 12, 13 (2012) and Mellor et al., J Hematol Oncol 6, 1 (2013). Furthermore, the in vivo protective activity of several therapeutic mAbs has been shown to depend on Fc-FcγR interactions, with Fc domain variants optimized for enhanced FcγR binding capacity exhibiting improved therapeutic outcome (Goede, V. et al. N Engl J Med 370, 1101-1110 (2014)). Given the diverse signaling activity of FcγRs (Bournazos et al., Annu Rev Immunol 35, 285-311 (2017)), engineering of the Fc domain to engage and activate specific classes of FcγRs has led to the development of IgG antibodies with improved effector activity. For example, the FDA-approved anti-CD20 mAb obinutuzumab, which is engineered for enhanced binding to the activating FcγR, FcγRIIIa, has been shown to exhibit superior therapeutic efficacy, compared to non-Fc engineered anti-CD20 mAbs (Goede, V. et al. N Engl J Med 370, 1101-1110 (2014)).

However, various challenges remain (Klein et al. 2012, MAbs. 4 (6): 653-663). In particular, the diversity of Fc receptors and their restricted expression on cells of the immune system has been demonstrated to impact on the range of responses that are associated with antibody-mediated activities. For example, the ability of an antibody to induce T cell responses has been shown to be dependent on engagement of dendritic cell activation Fc receptors, such as FcRIIA (DiLillo, et al., Cell 2015). Similarly, the activation of neutrophils by IgG antibodies require different Fc receptors than that of NK cells. In addition, as disclosed in this document, new modified IgG antibodies of this invention have half-lives equal to or greater than unmodified IgG1 in vivo. Thus, there is a need for Fc variants that are capable of the full range of low-affinity activation receptor engagement, with minimal engagement of the inhibitory Fc receptor, FcRIIB.

### SUMMARY OF INVENTION

Various embodiments described in this document address the above-mentioned unmet needs and/or other needs by providing human IgG Fc domain variants with improved effector function and half-lives, and uses thereof.

In one aspect, the invention relates to a polypeptide comprising an Fc variant of a human IgG1 Fc polypeptide. The Fc variant (i) comprises an Alanine (A) at position 236, a Leucine (L) at position 330, and a Glutamic acid (E) at position 332, and (ii) does not comprise an Aspartic acid (D) at position 239. The numbering is according to the EU index in Kabat. The polypeptide or the Fc variant may further comprise a Leucine (L) at position 428 and/or a Serine(S) at position 434. In some embodiments, the polypeptide or the Fc variant contains a Serine(S) at position 239. In some examples, the polypeptide or the Fc variant contains the sequence of SEQ ID NO: 2 or 3.

The above-mentioned polypeptide or Fc variant can be included as a part in an antibody or fusion protein (e.g., fused to Fv, sFv or other antibody variants as described below). Accordingly, within the scope of this invention is an antibody or fusion protein comprising the polypeptide or Fc variant described above. The antibody has specificity for any target molecule of interest. For example, the target molecule can be selected from the group consisting of a cytokine, a soluble or insoluble factor, a molecule expressed on a pathogen, a molecule expressed on cells, and a molecule expressed on cancer cells. The factors and molecules can be proteins and non-proteins, such as carbohydrates and lipids. The antibody can be selected from the group consisting of a chimeric antibody, a humanized antibody, or a human antibody. The above-described antibody can have one or more of the following features: (1) a higher binding affinity to hFcγRIIA, hFcγRIIIA, FcRn, or/and hFcγRIIIB as compared to a reference antibody having the sequence of SEQ ID NO: 1, (2) a longer serum half-life as compared to a reference antibody having the sequence of SEQ ID NO: 1 or 4, and (3) identical or better half-life as compared to an antibody having the sequence of SEQ ID NO:1. The above-described antibody is generally the same as the reference antibody except that the latter has a different Fc sequence, e.g., SEQ ID NO: 1 or 4. For example, the GAALIE variant (SEQ ID NO: 2) disclosed herein is unexpectedly more stable with a longer half-life than the GASDALIE variant (SEQ ID NO: 4).

Also within the scope of this invention are an isolated nucleic acid comprising a sequence encoding the polypeptide or antibody described above, an expression vector comprising the nucleic acid, and a host cell comprising the nucleic acid. The host cell can be used in a method of producing a recombinant polypeptide or an antibody. The method includes culturing the host cell in a medium under conditions permitting expression of a polypeptide or antibody encoded by the nucleic acid, and purifying the polypeptide or antibody from the cultured cell or the medium of the cell.

In another aspect, the invention provides a pharmaceutical formulation comprising (i) the polypeptide, antibody, or nucleic acid described above and (ii) a pharmaceutically acceptable carrier.

In another aspect, the invention provides a method of treating a disorder, such as an inflammatory disorder, a neoplastic disorder, or an infectious disease. The method includes administering to a subject in need thereof a therapeutically effective amount of the above-described polypeptide, antibody, or nucleic acid. Also within the scope of this invention are uses of the polypeptide, antibody, or nucleic acid in manufacturing a medicament for treating a disorder, such as an inflammatory disorder, a neoplastic disorder, or an infectious disease.

The details of one or more embodiments of the invention are set forth in the description below. Other features, objectives, and advantages of the invention will be apparent from the description and from the claims.

### BRIEF DESCRIPTION OF THE DRAWINGS

FIGS. 1A, 1B, 1C, and 1D (collectively “FIG. 1”) are diagrams showing in vivo half-life of the G236A/S239D/A330L/1332E (“GASDALIE”) Fc domain mutant in FcγR humanized (FcR+) (FIG. 1A and FIG. 1C) and in FcR deficient (FcR null) mice (FIG. 1B and FIG. 1D). An S239D/1332E (“SDIE”) variant was included as control. FIG. 1C and FIG. 1D show serum IgG levels of human IgG1 Fc variants 8 days following administration to FcγR humanized (FIG. 1C) and FcR deficient (FIG. 1D) mice.

FIGS. 2A and 2B (collectively “FIG. 2”) are diagrams showing the determination of in vivo half-life of Fc domain mutants in rhesus macaques. Wild-type (WT) human IgG1 (FIG. 2A) and a G236A/A330L/1332E/M428L/N434S (“GASDALIE LS”) (FIG. 2B) Fc domain variants of the 3BNC117 mAb were administered (i.v.; 20 mg/kg) to rhesus monkeys. IgG levels of human IgG1 were evaluated by ELISA at different time points following administration to rhesus monkeys to determine the antibody half-life (expressed as h).

FIGS. 3A and 3B (collectively “FIG. 3”) are tables showing binding affinity of Fc domain variants of human IgG1 for human FcγRs (FcγRIIa H131, FcγRIIa R131, FcγRIIb, FcγRIIIa V157, FcγRIIIa F157) determined by SPR analysis. FIG. 3A shows affinity measurements (KD (M)), and FIG. 3B shows fold increase in affinity over wild-type human IgG1. Variants tested: SDIE (S239D/1332E); GAIE (G236A/I332E); GAALIE (G236A/A330L/1332E); afucosylated (lacking branching fucose residue on the Fc-associated glycan).

FIG. 4 is a set of diagrams showing SPR sensorgrams of the binding of wild-type human IgG1 (left) and GAALIE (right) Fc domain variant for human FcγRs (FcγRIIa H131, FcγRIIa R131, FcγRIIb, FcγRIIIa V157, FcγRIIIa F157). Labels represent the analyte (FcγR) concentration (μM).

FIGS. 5A and 5B (collectively “FIG. 5”) are tables showing binding affinity of Fc domain variants of human IgG1 for mouse FcγRs determined by SPR analysis. FIG. 5A shows affinity measurements (KD (M)), and FIG. 5B shows fold increase in affinity over wild-type human IgG1. Variants tested: SDIE (S239D/1332E); GAIE (G236A/1332E); GAALIE (G236A/A330L/1332E); afucosylated (lacking branching fucose residue on the Fc-associated glycan).

FIG. 6 is a set of diagrams showing SPR sensorgrams of the binding of wild-type human IgG1 (left) and GAALIE (right) Fc domain variant for mouse FcγRs. Labels represent the analyte (FcγR) concentration (μM).

FIGS. 7A and 7B (collectively “FIG. 7”) are tables showing binding affinity of Fc domain variants of human IgG1 for rhesus FcγRs determined by SPR analysis. FIG. 7A shows affinity measurements (KD (M)), and FIG. 7B shows fold increase in affinity over wild-type human IgG1. Variants tested: SDIE (S239D/1332E); GAIE (G236A/I332E); GAALIE (G236A/A330L/1332E); afucosylated (lacking branching fucose residue on the Fc-associated glycan).

FIG. 8 is a set of diagrams showing SPR sensorgrams of the binding of wild-type human IgG1 (left) and GAALIE (right) Fc domain variant for rhesus FcγRs. Labels represent the analyte (FcγR) concentration (μM).

FIG. 9 is a diagram showing platelet depletion with 6A6 mAb Fc variants in FcγR humanized mice. Mice received Fc domain variants of the 6A6 mAb (SDIE (S239D/1332E); GAIE (G236A/I332E); GAALIE (G236A/A330L/1332E)). N297A (non-FcR binding variant) was included as control. Platelet numbers were analyzed at the indicated time points, and values represent the mean (±SEM) percentage change in platelet number relative to the prebleed at 0 h.

FIGS. 10A and 10B (collectively “FIG. 10”) are diagrams showing CD4+ cell depletion with GK1.5 mAb Fc variants in FcγR humanized mice. Mice received Fc domain variants (100 μg, i.p.) of the GK1.5 mAb (SDIE (S239D/1332E); GAIE (G236A/I332E); GAALIE (G236A/A330L/1332E)). GRLR (G236R/L328R; non-FcR binding variant) was included as control. CD4+ cell numbers were analyzed 24 h post mAb administration in blood (A) and spleen (B).

FIGS. 11A, 11B, 11C, and 11D (collectively “FIG. 11”) are diagrams showing CD20+ B-cell depletion with CAT mAb Fc variants in hCD20+/FcγR humanized mice. Mice received Fc domain variants (200 μg, i.p.) of the CAT mAb (SDIE (S239D/1332E); GAIE (G236A/I332E); GAALIE (G236A/A330L/1332E)). N297A (non-FcR binding variant) was included as control. CD20+ cell numbers and frequencies were analyzed 48 h post-mAb administration in blood (FIG. 11A and FIG. 11B) and spleen (FIG. 11C and FIG. 11D).

FIGS. 12A and 12B (collectively “FIG. 12”) are diagrams showing CD20+ B-cell depletion with 2B8 mAb Fc variants in hCD20+/ FcγR humanized mice. Mice received i.p. wild-type human IgG1 or GAALIE (G236A/A330L/1332E) variants of the anti-CD20 mAb 2B8 at the indicated dose. CD20+ frequencies (FIG. 12A) and cell numbers (FIG. 12B) were analyzed 48 h post-mAb administration in blood.

FIGS. 13A, 13B, and 13C (collectively “FIG. 13”) are diagrams showing in vivo half-life of Fc domain mutants in FcR deficient (FcR null) (FIG. 13A) and FcγR humanized mice (FcR+) (FIG. 13B). Fc domain mutants of human IgG1 included: SDIE (S239D/1332E), GAIE (G236A/1332E), and GAALIE (G236A/A330L/1332E). FIG. 13C shows IgG levels of human IgG1 at different time points following administration to FcγR humanized mice.

FIGS. 14A and 14B (collectively “FIG. 14”) are diagrams showing the determination of in vivo half-life of Fc domain mutants in rhesus macaques. Wild-type (WT) human IgG1 (FIG. 14A) and GAALIE (G236A/A330L/1332E) (FIG. 14B) Fc domain variants of the 3BNC117 mAb were administered (i.v.; 20 mg/kg) to rhesus monkeys. IgG levels of human IgG1 were evaluated by ELISA at different time points following administration to rhesus monkeys to determine the antibody half-life (expressed as h).

FIGS. 15A and 15B (collectively “FIG. 15”) are diagrams showing CD20+ B-cell depletion with 2B8 mAb Fc variants in rhesus macaques. Wild-type human IgG1 or GAALIE (G236A/A330L/1332E) variants of the anti-CD20 mAb 2B8 were administered to rhesus monkeys (i.v.) at 0.05 mg/kg. CD20+ frequencies (FIG. 15A) and cell numbers (FIG. 15B) were analyzed in blood at various time points before and after antibody administration.

FIG. 16 shows protein sequences of the constant regions of human IgG1 (wild-type and Fc domain variants). Amino acid substitutions for each variant are underlined. Residue numbering follows the EU numbering system.

FIG. 17 is a diagram showing protein Tm of the various Fc domain mutants determined by the Thermal Shift Assay. Fc domain mutants of human IgG1 included: SDIE (S239D/1332E), GAIE (G236A/I332E), GAALIE (G236A/A330L/1332E), and GASDALIE (G236A/S239D/A330L/1332E). These mutants were combined with the LS mutation (M428L/N434S), which increases the affinity of human IgG1 to FcRn.

FIG. 18 is a table showing binding affinity of Fc domain variants of human IgG1 for human FcRn/B2 microglobulin at pH 6.0 as determined by SPR analysis. Affinity measurements (KD (M)) and fold increase in affinity over wild-type human IgG1 are presented. Fc domain mutants of human IgG1 included: SDIE (S239D/1332E), GAIE (G236A/1332E), and GAALIE (G236A/A330L/1332E). These mutants were combined with the LS mutation (M428L/N434S).

FIG. 19 is a set of diagrams showing SPR sensorgrams of the binding of Fc domain variants to human FcRn/B2 microglobulin at pH 6.0. Labels represent the analyte (FcRn) concentration (nM). Fc domain mutants of human IgG1 included: LS (M428L/N434S), GAALIE (G236A/A330L/1332E), and GAALIE LS (G236A/A330L/1332E/M428L/N434S).

FIG. 20 is a set of diagrams showing SPR sensorgrams of the binding of Fc domain variants to human FcRn/B2 microglobulin at pH 7.4. Labels represent the analyte (FcRn) concentration (nM). Fc domain mutants of human IgG1 included: LS (M428L/N434S), GAALIE (G236A/A330L/1332E), and GAALIE LS (G236A/A330L/1332E/M428L/N434S).

FIGS. 21A, 21B, and 21C (collectively “FIG. 21”) are a set of diagrams showing in vivo half-life of Fc domain mutants in FcRn/FcγR humanized mice. Fc domain mutants of human IgG1 included: LS (M428L/N434S), GAALIE (G236A/A330L/1332E), and GAALIE LS (G236A/A330L/1332E/M428L/N434S). FIG. 21A and FIG. 21B show IgG levels of human IgG1 at different time points following administration to FcRn/FcγR humanized mice. FIG. 21C shows calculated half-life of Fc domain variants in FcRn/FcγR humanized mice.

FIG. 22 is a diagram showing platelet depletion with 6A6 mAb Fc variants in FcRn/FcγR humanized mice. Mice received Fc domain variants of the 6A6 mAb (8 μg; i.v.) (LS (M428L/N434S), GAALIE (G236A/A330L/1332E), and GAALIE LS (G236A/A330L/1332E/M428L/N434S)). N297A (non-FcR binding variant) was included as control. Platelet numbers were analyzed at the indicated time points, and values represent the mean (+SEM) percentage change in platelet number relative to the prebleed at 0 h.

FIGS. 23A, 23B, 23C, and 23D (collectively “FIG. 23”) are diagrams showing that sLeA-targeting Abs with a hIgG1 Fc promote tumor clearance enhanced by engaging activating human FcγRs. FcγR-humanized mice were inoculated IV with 5*105 B16-FUT3 tumor cells. 100 μg of anti-sLeA Abs or isotype-matched control Abs were administered IP on days 1,4,7 and 11. 14 days post-inoculation, mice were euthanized, lungs were excised and fixed, and metastatic foci were counted. n≥5/group. *p<0.05, **p<0.01, ***p<0.001. ****p<0.0001. FIGS. 23A and 23B show that anti-sLeA hIgG1 Abs inhibit lung colonization of sLeA+ tumor cells. Mice were treated with 100 μg of anti-sLeA Abs (5B1-hIgG1 or 7E3-hIgG1) or isotype-matched control Abs. FIG. 23A shows an aggregated analysis of the data obtained for all mice from a representative experiment, and FIG. 23B shows representative images of three excised lungs from each group. FIG. 23B also shows that Fc-engineered Anti-sLeA Ab variants demonstrate superior anti-tumor efficacy-mice were treated with 100 μg of anti-sLeA Abs (clones 5B1 or 7E3, hIgG1 or hIgG1-GAALIE with G236A/A330L/1332E mutations) or isotype-matched control Abs. FIG. 23C shows an aggregated analysis of the data obtained for all mice from two separate experiments (first experiment-▪, second experiment-▴), while FIG. 23D shows representative images of excised lungs from mice treated with 5B1 Abs.

FIGS. 24A, 24B, and 24C (collectively “FIG. 24”) are diagrams showing that engagement of either hFcRIIA or hFcRIIIA is necessary and sufficient for Ab-mediated tumor clearance. FIG. 24A shows the relative binding affinity of hIgG1 Fc variants to human FcRs-affinity as determined by SPR studies. FIG. 24B shows 5B1-hIgG1 Abs with enhanced binding affinity to hFcRIIA, or hFcRIIIA or both, demonstrating a superior anti-tumor effect. FcγR-humanized mice were inoculated IV with 5*105 B16-FUT3 tumor cells. 100 μg of anti-sLeA Abs (5B1-hIgG1, 5B1-hIgG1-GA with a G236A mutation, 5B1-hIgG1-ALIE with A330L/1332E mutations or 5B1-hIgG1-GAALIE with G236A/A330L/1332E mutations) or isotype-matched control Abs were administered IP on days 1,4,7 and 11. FIG. 24C shows hFcRIIA or hFcRIIIA engagement, which is essential for efficient tumor clearance of sLeA+ tumors. FcR-null (γ chain KO), FcγR-humanized, hFcRIIA/IIBtransgenic, and hFcRIIIA/IIIB-transgenic mice were inoculated IV with 5*105 B16-FUT3 tumor cells. 100 μg of anti-sLeA Abs (5B1-hIgG1-GAALIE with G236A/A330L/1332E mutations) or isotype-matched control Abs were administered IP on days 1,4,7 and 11. For panels B+C, 14 days post-inoculation, mice were euthanized, lungs were excised and fixed, and metastatic foci were counted. n≥6/group. *p<0.05, ***p<0.001. ****p<0.0001.

### DETAILED DESCRIPTION OF THE INVENTION

This document describes human IgG Fc domain variants with improved effector function and uses thereof. As described herein, antibodies or fusion proteins having the IgG Fc domain variants have increased binding to activation Fc receptors and half-lives equal to or greater than unmodified IgG1 antibodies in vivo.

The Fc regions or constant regions of antibodies interact with cellular binding partners to mediate antibody function and activity, such as antibody-dependent effector functions and complement activation. For IgG type antibodies, the binding sites for complement Clq and Fc receptors (FcγRs) are located in the CH2 domain of the Fc region. The co-expression of activating and inhibitory FcRs on different target cells modulates antibody-mediated immune responses. In addition to their involvement in the efferent phase of an immune response, FcRs are also important for regulating B cell and dendritic cell (DC) activation. For example, in the case of IgG type antibodies, different classes of FcγR mediate various cellular responses, such as phagocytosis by macrophages, antibody-dependent cell-mediated cytotoxicity by NK cells, and degranulation of mast cells. Each FcγR displays different binding affinities and IgG subclass specificities. Lectin receptors also play a role. For example, DC-SIGN has been shown to play a role in the anti-inflammatory activity of Fc, e.g., in IVIG (see, e.g., US20170349662, WO2008057634, and WO2009132130).

As described herein, the biological activity of an antibody/immunoglobulin can be manipulated, altered, or controlled by introducing mutations or altering certain amino acids of the Fc region. Biological activities that can be manipulated, altered, or controlled in light of the present disclosure include, for example, one or more of: Fc receptor binding, Fc receptor affinity, Fc receptor specificity, complement activation, signaling activity, targeting activity, effector function (such as programmed cell death or cellular phagocytosis), half-life, clearance, and transcytosis.

### I. Definitions

The terms “peptide,” “polypeptide,” and “protein” are used herein interchangeably to describe the arrangement of amino acid residues in a polymer. A peptide, polypeptide, or protein can be composed of the standard 20 naturally occurring amino acid, in addition to rare amino acids and synthetic amino acid analogs. They can be any chain of amino acids, regardless of length or post-translational modification (for example, glycosylation or phosphorylation).

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

An “isolated” peptide, polypeptide, or protein refers to a peptide, polypeptide, or protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide/protein described in the invention can be produced by recombinant DNA techniques, purified from a transgenic animal source, or by chemical methods. A functional equivalent of IgG Fc refers to a polypeptide derivative of IgG Fc, e.g., a protein having one or more point mutations, insertions, deletions, truncations, a fusion protein, or a combination thereof. It retains substantially the activity of the IgG Fc, i.e., the ability to bind to the respective receptor and trigger the respective cellular response. The isolated polypeptide can contain SEQ ID NO: 2 or 3. In general, the functional equivalent is at least 75% (e.g., any number between 75% and 100%, inclusive, e.g., 70%, 80%, 85%, 90%, 95%, 96%, 97%, 98%, and 99%) identical to SEQ ID NO: 2 or 3.

An “antigen” refers to a substance that elicits an immunological reaction or binds to the products of that reaction. The term “epitope” refers to the region of an antigen to which an antibody or T cell binds.

As used herein, “antibody” is used in the broadest sense and specifically covers monoclonal antibodies (including full-length monoclonal antibodies), polyclonal antibodies, multispecific antibodies (e.g., bispecific antibodies), and antibody fragments so long as they exhibit the desired biological activity. The term “antibody” (Ab) as used herein includes monoclonal antibodies, polyclonal antibodies, multispecific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2, IgG3, IgG4, IgM, IgA, IgD, IgE and IgM); and biologically relevant fragment or specific binding member thereof, including but not limited to Fab, F(ab′)2, Fv, and scFv (single chain or related entity). It is understood in the art that an antibody is a glycoprotein comprising at least two heavy (H) chains and two light (L) chains inter-connected by disulfide bonds, or an antigen-binding portion thereof. A heavy chain is comprised of a heavy chain variable region (VH) and a heavy chain constant region (CH1, CH2, and CH3). A light chain is comprised of a light chain variable region (VL) and a light chain constant region (CL). The variable regions of both the heavy and light chains comprise framework regions (FWR) and complementarity determining regions (CDR). The four FWR regions are relatively conserved while CDR regions (CDR1, CDR2, and CDR3) represent hypervariable regions and are arranged from NH2 terminus to the COOH terminus as follows: FWR1, CDR1, FWR2, CDR2, FWR3, CDR3, and FWR4. The variable regions of the heavy and light chains contain a binding domain that interacts with an antigen while, depending on the isotype, the constant region(s) may mediate the binding of the immunoglobulin to host tissues or factors. Also included in the definition of “antibody” as used herein are chimeric antibodies, humanized antibodies, and recombinant antibodies, human antibodies generated from a transgenic non-human animal, as well as antibodies selected from libraries using enrichment technologies available to the artisan.

As used herein, “antibody fragments,” may comprise a portion of an intact antibody, generally including the antigen binding and variable region of the intact antibody and/or the Fc region of an antibody which retains FcR binding capability. Examples of antibody fragments include linear antibodies; single-chain antibody molecules; and multispecific antibodies formed from antibody fragments. Preferably, the antibody fragments retain the entire constant region of an IgG heavy chain and include an IgG light chain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler and Milstein, Nature, 256, 495-497 (1975), which is incorporated herein by reference, or may be made by recombinant DNA methods (see, e.g., U.S. Pat. No. 4,816,567, which is incorporated herein by reference). The monoclonal antibodies may also be isolated from phage antibody libraries using the techniques described in Clackson et al., Nature, 352, 624-628 (1991) and Marks et al., J Mol Biol, 222, 581-597 (1991), for example, each of which is incorporated herein by reference.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; Morrison et al., Proc Natl Acad Sci USA, 81, 6851-6855 (1984); Neuberger et al., Nature, 312, 604-608 (1984); Takeda et al., Nature, 314, 452-454 (1985); International Patent Application No. PCT/GB85/00392, each of which is incorporated herein by reference).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody optionally also will comprise at least a portion of an immunoglobulin constant region (Fc), typically that of a human immunoglobulin. For further details, see Jones et al., Nature, 321, 522-525 (1986); Riechmann et al., Nature, 332, 323-329 (1988); Presta, Curr Op Struct Biol, 2, 593-596 (1992); U.S. Pat. No. 5,225,539, each of which is incorporated herein by reference.

“Human antibodies” refer to any antibody with fully human sequences, such as might be obtained from a human hybridoma, human phage display library or transgenic mouse expressing human antibody sequences.

The term “variable” refers to the fact that certain segments of the variable (V) domains differ extensively in sequence among antibodies. The V domain mediates antigen binding and defines the specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta sheet configuration, connected by three hypervariable regions, which form loops connecting, and in some cases forming part of, the beta sheet structure. The hypervariable regions in each chain are held together in close proximity by the FRs and, with the hypervariable regions from the other chain, contribute to the formation of the antigen-binding site of antibodies (see, for example, Kabat et al., Sequences of Proteins of Immunological Interest, 5th Ed. Public Health Service, National Institutes of Health, Bethesda, Md. (1991)).

The term “hypervariable region” as used herein refers to the amino acid residues of an antibody that are responsible for antigen binding. The hypervariable region generally comprises amino acid residues from a “complementarity determining region” (“CDR”).

“Fv” is the minimum antibody fragment that contains a complete antigen-recognition and antigen-binding site. This fragment contains a dimer of one heavy-and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable region (or half of an Fv comprising only three CDRs specific for an antigen) has the ability to recognize and bind antigen, although at a lower affinity than the entire binding site.

“Single-chain Fv” (“sFv” or “scFv”) are antibody fragments that comprise the VH and VL antibody domains connected into a single polypeptide chain. The sFv polypeptide can further comprise a polypeptide linker between the VH and VL domains that enables the sFv to form the desired structure for antigen binding. For a review of sFv, see, for example, Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments with short linkers (about 5-10 residues) between the VH and VL domains such that inter-chain but not intra-chain pairing of the V domains is achieved, resulting in a bivalent fragment, i.e., a fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

Domain antibodies (dAbs), which can be produced in fully human form, are the smallest known antigen-binding fragments of antibodies, ranging from about 11 kDa to about 15 kDa. DAbs are the robust variable regions of the heavy and light chains of immunoglobulins (VH and VL, respectively). They are highly expressed in microbial cell culture, show favorable biophysical properties including, for example, but not limited to, solubility and temperature stability, and are well suited to selection and affinity maturation by in vitro selection systems such as, for example, phage display. DAbs are bioactive as monomers and, owing to their small size and inherent stability, can be formatted into larger molecules to create drugs with prolonged serum half-lives or other pharmacological activities. Examples of this technology have been described in, for example, WO9425591 for antibodies derived from Camelidae heavy chain Ig, as well in US20030130496 describing the isolation of single domain fully human antibodies from phage libraries.

Fv and sFv are the only species with intact combining sites that are devoid of constant regions. Thus, they are suitable for reduced nonspecific binding during in vivo use. sFv fusion proteins can be constructed to yield fusion of an effector protein at either the amino or the carboxy terminus of an sFv. See, for example, Antibody Engineering, ed. Borrebaeck, supra. The antibody fragment also can be a “linear antibody,” for example, as described in U.S. Pat. No. 5,641,870. Such linear antibody fragments can be monospecific or bispecific.

As used herein, the term “Fc fragment” or “Fc region” is used to define a C-terminal region of an immunoglobulin heavy chain. Such an Fc region is the tail region of an antibody that interacts with Fc receptors and some proteins of the complement system. The Fc region may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. A native sequence Fc region comprises an amino acid sequence identical to the amino acid sequence of an Fc region found in nature. A variant Fc region as appreciated by one of ordinary skill in the art comprises an amino acid sequence which differs from that of a native sequence Fc region by virtue of at least one “amino acid modification.”

In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site. Glycosylation of the Fc fragment is important for Fc receptor-mediated activity. The N-glycans attached to this site are predominantly core-fucosylated biantennary structures of the complex type. In addition, small amounts of these N-glycans also bear bisecting GlcNAc and α-2,6 linked sialic acid residues. See, e.g., US20170349662, US20080286819, US20100278808, US20100189714, US2009004179, 20080206246, 20110150867, and WO2013095966, each of which is incorporated herein by reference.

A “native sequence Fc region” comprises an amino acid sequence identical to the amino acid sequence of an Fc region found in nature. A “variant Fc region” or “Fc variant” or “Fc domain variant” as appreciated by one of ordinary skill in the art comprises an amino acid sequence which differs from that of a native sequence Fc region by virtue of at least one “amino acid modification.” Preferably, the variant Fc region has at least one amino acid substitution compared to a native sequence Fc region or to the Fc region of a parent polypeptide, e.g., from about one to about ten amino acid substitutions, and preferably from about one to about six, five, four, three, or two amino acid substitutions in a native sequence Fc region or in the Fc region of the parent polypeptide. The variant Fc region herein will preferably possess at least about 75 or 80% homology with a native sequence Fc region and/or with an Fc region of a parent polypeptide, and more preferably at least about 90% homology therewith, more preferably at least about 95% homology therewith, even more preferably, at least about 96%, 97%, 98%, or 99% homology therewith. The term “native” or “parent” refers to an unmodified polypeptide comprising an Fc amino acid sequence. The parent polypeptide may comprise a native sequence Fc region or an Fc region with pre-existing amino acid sequence modifications (such as additions, deletions and/or substitutions).

The terms “Fc receptor” or “FcR” are used to describe a receptor that binds to the Fc region of an antibody. An Fc receptor is a protein found on the surface of certain cells-including, among others, B lymphocytes, follicular dendritic cells, natural killer cells, macrophages, neutrophils, eosinophils, basophils, and mast cells-that contribute to the protective functions of the immune system. Its name is derived from its binding specificity for the Fc region (fragment crystallizable region) of an antibody.

Several antibody functions are mediated by Fc receptors. For example, Fc receptors bind to antibodies that are attached to infected cells or invading pathogens. Their activity stimulates phagocytic or cytotoxic cells to destroy microbes or infected cells by antibody-mediated phagocytosis or antibody-dependent cell-mediated cytotoxicity. It was also known in the art that the Fc region of an antibody ensures that each antibody generates an appropriate immune response for a given antigen, by binding to a specific class of Fc receptors, and other immune molecules, such as complement proteins. FcRs are defined by their specificity for immunoglobulin isotypes: Fc receptors for IgG antibodies are referred to as FcγR, for IgE as FcεFR, for IgA as FcαR and so on. Surface receptors for immunoglobulin G are present in two distinct classes-those that activate cells upon their crosslinking (“activation FcRs”) and those that inhibit activation upon co-engagement (“inhibitory FcRs”).

In mammalian species, multiple different classes of IgG Fc-receptors have been defined: FcγRI (CD64), FcγRII (CD32), FcγRIII (CDI6) and FcγIV in mice, for example, and FcRI, FcRIIA, B, C, FcRIIIA and B in human, for example. Whereas FcγRI displays high affinity for the antibody constant region and restricted isotype specificity, FcγRII and FcγRIII have low affinity for the Fc region of IgG but a broader isotype binding pattern (Ravetch and Kinet, 1991; Hulett and Hogarth, Adv Immunol 57, 1-127 (1994)). FcγRIV is a recently identified receptor, conserved in all mammalian species with intermediate affinity and restricted subclass specificity (Mechetina et al., Immunogenetics 54, 463-468 (2002); Davis et al., Immunol Rev 190, 123-136 (2002); Nimmerjahn et al., Immunity 23, 41-51 (2005)).

Functionally there are two different classes of Fc-receptors: the activation and the inhibitory receptors, which transmit their signals via immunoreceptor tyrosine-based activation (ITAM) or inhibitory motifs (ITIM), respectively (Ravetch, in Fundamental Immunology W. E. Paul, Ed. (Lippincott-Raven, Philadelphia, (2003); Ravetch and Lanier, Science 290, 84-89 (2000). The paired expression of activating and inhibitory molecules on the same cell is the key for the generation of a balanced immune response. Additionally, it has been appreciated that the IgG Fc-receptors show significant differences in their affinity for individual antibody isotypes rendering certain isotypes more strictly regulated than others (Nimmerjahn et al., 2005).

In one embodiment of the invention, FcR is a native sequence human FcR. In another embodiment, FcR, including human FcR, binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review in Daron, Annu Rev Immunol, 15, 203-234 (1997); FcRs are reviewed in Ravetch and Kinet, Annu Rev Immunol, 9, 457-92 (1991); Capel et al., Immunomethods, 4, 25-34 (1994); and de Haas et al, J Lab Clin Med, 126, 330-41 (1995), Nimmerjahn and Ravetch 2006, Ravetch Fc Receptors in Fundamental Immunology, ed William Paul 5th Ed. each of which is incorporated herein by reference).

The term “pharmaceutical composition” refers to the combination of an active agent with a carrier, inert or active, making the composition especially suitable for diagnostic or therapeutic use in vivo or ex vivo.

As used herein, “pharmaceutically acceptable carrier” includes any and all solvents, dispersion media, coatings, antibacterial and antifungal agents, isotonic and absorption delaying agents, and the like that are physiologically compatible. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. Preferably, the carrier is suitable for intravenous, intramuscular, subcutaneous, parenteral, spinal or epidermal administration (e.g., by injection or infusion). The therapeutic compounds may include one or more pharmaceutically acceptable salts. A “pharmaceutically acceptable salt” refers to a salt that retains the desired biological activity of the parent compound and does not impart any undesired toxicological effects (see, e.g., Berge, S. M., et al. (1977) J. Pharm. Sci. 66:1-19).

The term “cytotoxic agent” as used herein refers to a substance that inhibits or prevents the function of cells and/or causes the destruction of cells. The term is intended to include radioactive isotopes (e.g. At211, Il31, Il25, Y90, Re186, Re188, Sm153, Bi212, P32 and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, see, e.g., Agnew Chem. Intl. Ed. Engl. 33:183-186 (1994); dynemicin, including dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®.; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

The terms “prevent,” “preventing,” “prevention,” “prophylactic treatment” and the like refer to reducing the probability of developing a disorder or condition in a subject, who does not have, but is at risk of or susceptible to developing a disorder or condition.

A “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental, non-human animal or animal suitable as a disease model.

An “effective amount” refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment. A therapeutically effective amount of a combination to treat a neoplastic condition is an amount that will cause, for example, a reduction in tumor size, a reduction in the number of tumor foci, or slow the growth of a tumor, as compared to untreated animals.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example, “about 1” may also mean from 0.5 to 1.4.

### II. Polypeptides and Antibodies

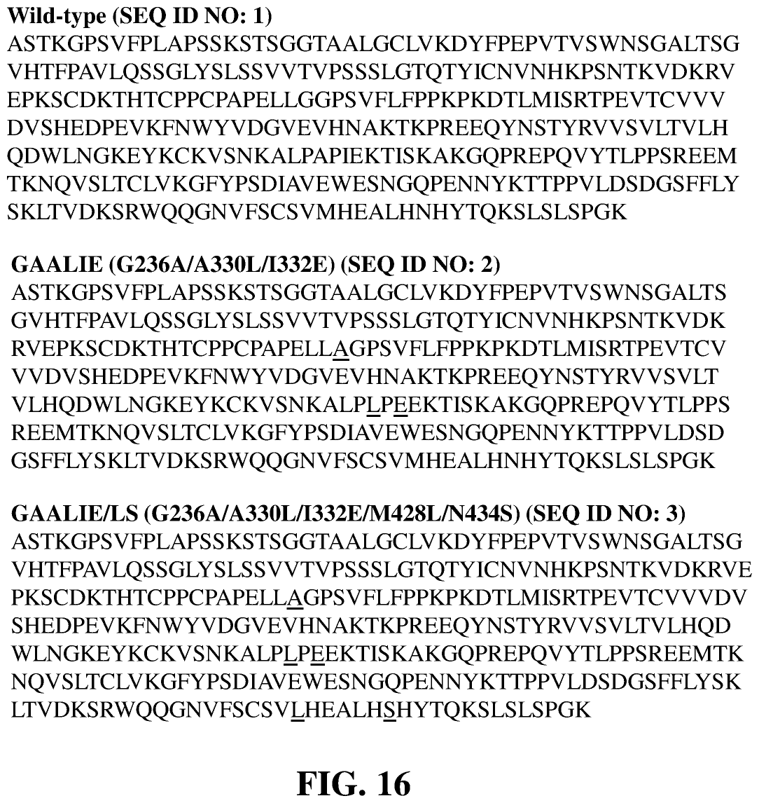

As disclosed herein, this invention provides isolated polypeptides having sequences of variants of human IgG Fc (such as hIgG1 Fc). In one embodiment, the Fc region includes one or more substitutions of the hIgG1 Fc amino acid sequence. While not limited thereto, exemplary IgG1 Fc regions are provided below and in FIG. 16. In the sequences, amino acid residues at positions 236, 239, 330, 332, 428, and 434 in each sequence are in bold while amino acid substitutions underlined. Residue numbering follows the EU numbering system and the first residue, A, corresponds to position 118 under the EU numbering system.

Wild-type:
(SEQ ID NO: 1)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
GAALIE (G236A/A330L/I332E):
(SEQ ID NO: 2)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLAGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPLPEEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
GAALIE/LS (G236A/A330L/I332E/M428L/N434S):
(SEQ ID NO: 3)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLAGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPLPEEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVLHEALHSHYTQKSLSLSPGK
GASDALIE (G236A/A330L/I332E):
(SEQ ID NO: 4)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLAGPDVFLFPPKPKDTLMISRTPEVTCVVV
DVSHEDPEVKFNWYVDGVEVHNAKTKPREEQYNSTYRVVSVLTVLHQDW
LNGKEYKCKVSNKALPLPEEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

The amino acid composition of the polypeptide described herein may vary without disrupting the ability of the polypeptide to bind to the respective receptor and trigger the respective cellular response. For example, it can contain one or more conservative amino acid substitutions. A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It retains substantially the activity of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent (e.g., SEQ ID NO: 1, 2, 3, or 4). Accordingly, within the scope of this invention are Fc regions having one or more point mutations, insertions, deletions, truncations, a fusion protein (e.g., an Fv, sFv or other antibody variants as described below), or a combination thereof, as well as heavy chains or antibodies having the variant Fc regions.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a BLOSUM 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul et al. (1990) J. Mol. Biol. 215:403-10. BLAST protein searches can be performed with the XBLAST program, score=50, wordlength=3 to obtain amino acid sequences homologous to the molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. (See www.ncbi.nlm.nih.gov).

As used herein, the term “conservative modifications” refers to amino acid modifications that do not significantly affect or alter the binding characteristics of the antibody containing the amino acid sequence. Such conservative modifications include amino acid substitutions, additions, and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

A “conservative amino acid substitution” is one in which the amino acid residue is replaced with an amino acid residue having a similar side chain. Thus, a predicted nonessential amino acid residue in, e.g., SEQ ID NO: 2 or 3, is preferably replaced with another amino acid residue from the same side chain family. Alternatively, mutations can be introduced randomly along all or part of the sequences, such as by saturation mutagenesis, and the resultant mutants can be screened for the ability to bind to the respective receptor and trigger the respective cellular response to identify mutants that retain the activity as described below in the examples. Examples of conservative amino acid substitutions at positions other than positions 236, 239, 330, 332, 428, and 434 can be found in U.S. Pat. Nos. 9,803,023, 9,663,582, and 20,170,349662, the contents of which are incorporated herein.

A polypeptide as described in this invention can be obtained as a recombinant polypeptide. To prepare a recombinant polypeptide, a nucleic acid encoding it (e.g., SEQ ID NO: 2 or 3) can be linked to another nucleic acid encoding a fusion partner, e.g., glutathione-s-transferase (GST), 6x-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention.

Variant antibodies having the above-described Fc variants are within the scope of the invention. Further variants of the antibody sequences having improved affinity can be obtained using methods known in the art and are included within the scope of the invention. For example, amino acid substitutions can be used to obtain antibodies with further improved affinity. Alternatively, codon optimization of the nucleotide sequence can be used to improve the efficiency of translation in expression systems for the production of the antibody.

In certain embodiments, an antibody of the invention comprises a heavy chain variable region comprising CDR1, CDR2 and CDR3 sequences, and a light chain variable region comprising CDR1, CDR2, and CDR3 sequences. One or more of these CDR sequences comprise specified amino acid sequences based on the preferred antibodies described herein, or conservative modifications thereof, and wherein the antibodies retain the desired functional properties (e.g., neutralizing a pathogen such as multiple HIV-1 viral strains). Similarly, an antibody of the invention can comprise an Fc region of the preferred antibodies described herein, e.g., SEQ ID NO: 2 or 3, a section thereof, or conservative modifications thereof. One or more amino acid residues within the CDR or non-CDR regions of an antibody of the invention can be replaced with other amino acid residues from the same side chain family, and the altered antibody can be tested for retained function using the functional assays described herein. In the same vein, the variant Fc region described herein can have one or more conservative amino acid substitutions.

Other modifications of the antibody are contemplated herein. For example, the antibody can be linked to a cytotoxic agent, a chemotherapeutic agent, or to one of a variety of nonproteinaceous polymers, for example, polyethylene glycol, polypropylene glycol, polyoxyalkylenes, or copolymers of polyethylene glycol and polypropylene glycol. The antibody also can be entrapped in microcapsules prepared, for example, by coacervation techniques or by interfacial polymerization (for example, hydroxymethylcellulose or gelatin-microcapsules and poly-(methyl methacrylate) microcapsules, respectively), in colloidal drug delivery systems (for example, liposomes, albumin microspheres, microemulsions, nanoparticles and nanocapsules), or in macroemulsions. Such techniques are disclosed in, for example, Remington's Pharmaceutical Sciences, 16th edition, Oslo, A., Ed., (1980).

In certain embodiments, antibodies of the described invention are bispecific and can bind to two different epitopes of a single antigen. Other such antibodies can combine a first antigen binding site with a binding site for a second antigen. Bispecific antibodies also can be used to localize cytotoxic agents to infected cells. Bispecific antibodies can be prepared as full-length antibodies or antibody fragments (for example, F(ab′)2 bispecific antibodies). See, for example, WO 96/16673, U.S. Pat. No. 5,837,234, WO98/02463, U.S. Pat. No. 5,821,337, and Mouquet et al., Nature. 467, 591-5 (2010).

Methods for making bispecific antibodies are known in the art. Traditional production of full-length bispecific antibodies is based on the co-expression of two immunoglobulin heavy chain-light chain pairs, where the two chains have different specificities (see, for example, Millstein et al., Nature, 305:537-539 (1983)). Similar procedures are disclosed in, for example, WO 93/08829, Traunecker et al., EMBO J., 10:3655-3659 (1991) and see also; Mouquet et al., Nature. 467, 591-5 (2010). Techniques for generating bispecific antibodies from antibody fragments also have been described in the literature. For example, bispecific antibodies can be prepared using chemical linkage. See Brennan et al., Science, 229:81 (1985).

Typically, the antibodies used or described in the invention can be produced using conventional hybridoma technology or made recombinantly using vectors and methods available in the art. Human antibodies also can be generated by in vitro activated B cells (see, for example, U.S. Pat. Nos. 5,567,610 and 5,229,275). General methods in molecular genetics and genetic engineering useful in the present invention are described in the current editions of Molecular Cloning: A Laboratory Manual (Sambrook, et al., Molecular Cloning: A Laboratory Manual (Fourth Edition) Cold Spring Harbor Lab. press, 2012), Gene Expression Technology (Methods in Enzymology, Vol. 185, edited by D. Goeddel, 1991. Academic Press, San Diego, CA), “Guide to Protein Purification” in Methods in Enzymology (M. P. Deutscher et al. (1990) Academic Press, Inc.); PCR Protocols: A Guide to Methods and Applications (Innis et al. 1990. Academic Press, San Diego, CA), Culture of Animal Cells: A Manual of Basic Technique, 2nd Ed. (R. I. Freshney. 1987. Liss, Inc. New York, NY), and Gene Transfer and Expression Protocols, pp. 109-128, ed. E. J. Murray, The Humana Press Inc., Clifton, N.J.). Reagents, cloning vectors, and kits for genetic manipulation are available from commercial vendors such as BioRad, Stratagene, Invitrogen, ClonTech and Sigma-Aldrich Co.

Other techniques that are known in the art for the selection of antibody from libraries using enrichment technologies, including but not limited to phage display, ribosome display (Hanes and Pluckthun, 1997, Proc. Nat. Acad. Sci. 94:4937-4942), bacterial display (Georgiou, et al., 1997, Nature Biotechnology 15:29-34) and/or yeast display (Kieke, et al., 1997, Protein Engineering 10:1303-1310) may be utilized as alternatives to previously discussed technologies to select single chain antibodies. Single-chain antibodies are selected from a library of single chain antibodies produced directly utilizing filamentous phage technology. Phage display technology is known in the art (e.g., see technology from Cambridge Antibody Technology (CAT)) as disclosed in U.S. Pat. Nos. 5,565,332; 5,733,743; 5,871,907; 5,872,215; 5,885,793; 5,962,255; 6,140,471; 6,225,447; 6,291650; 6,492,160; 6,521,404; 6,544,731; 6,555,313; 6,582,915; 6,593, 081, as well as other U.S. family members, or applications which rely on priority filing GB 9206318, filed 24 May 1992; see also Vaughn, et al. 1996, Nature Biotechnology 14:309-314). Single chain antibodies may also be designed and constructed using available recombinant DNA technology, such as a DNA amplification method (e.g., PCR), or possibly by using a respective hybridoma cDNA as a template

Human antibodies also can be produced in transgenic animals (for example, mice) that are capable of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice results in the production of human antibodies upon antigen challenge. See, for example, Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); U.S. Pat. No. 5,545,807; and WO 97/17852. Such animals can be genetically engineered to produce human antibodies comprising a polypeptide of the described invention.

Any known monoclonal antibody may benefit from the Fc region variants and modifications disclosed in present disclosure by fusing its antigen-binding section to a Fc region/domain variant described herein. Examples of a known therapeutic monoclonal antibody may include any of the following, non-limiting antibodies: 3F8, 8H9, Abagovomab, Abciximab, Abituzumab, Abrilumab, Actoxumab, Adalimumab, Adecatumumab, Aducanumab, Afasevikumab, Afelimomab, Afutuzumab, Alacizumab pegol, ALD518, Alemtuzumab, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Carlumab, Carotuximab, Catumaxomab, CBR96-doxorubicin immunoconjugate, Cedelizumab, Cergutuzumab amunaleukin, Certolizumab pegol, Cetuximab, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Crotedumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMAB362, Imalumab, Imciromab, Imgatuzumab, Inclacumab, Indatuximab ravtansine, Indusatumab vedotin, Inebilizumab, Infliximab, Inolimomab, Inotuzumab ozogamicin, Intetumumab, Ipilimumab, Iratumumab, Isatuximab, Itolizumab, Ixekizumab, Keliximab, Labetuzumab, Lampalizumab, Lanadelumab, Landogrozumab, Laprituximab emtansine, Lebrikizumab, Lemalesomab, Lendalizumab, Lenzilumab, Lerdelimumab, Lexatumumab, Libivirumab, Lifastuzumab vedotin, Ligelizumab, Lilotomab satetraxetan, Lintuzumab,Lirilumab, Lodelcizumab, Lokivetmab, Lorvotuzumab mertansine, Lucatumumab, Lulizumab pegol, Lumiliximab, Lumretuzumab, MABp1, Mapatumumab, Margetuximab, Maslimomab, Matuzumab, Mavrilimumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, SGN-CD19A, SGN-CD33A, Sibrotuzumab, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tamtuvetmab, Tanezumab, Taplitumomab paptox, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tigatuzumab, Tildrakizumab, Timolumab, Tisotumab vedotin, TNX-650, Tocilizumab, Toralizumab, Tosatoxumab, Tositumomab, Tovetumab, Tralokinumab, Trastuzumab, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, Zolimomab aritox, and combinations thereof.

The targets may comprise any of the following, non-limiting targets: β-amyloid, 4-1BB, 5AC, 5T4, α-fetoprotein, angiopoietin, AOC3, B7-H3, BAFF, c-MET, c-MYC, C242 antigen, C5, CA-125, CCL11, CCR2, CCR4, CCR5, CD4, CD8, CD11, CD18, CD125, CD140a, CD127, CD15, CD152, CD140, CD19, CD2, CD20, CD22, CD23, CD25, CD27, CD274, CD276, CD28, CD3, CD30, CD33, CD37, CD38, CD4, CD40, CD41, CD44, CD47, CD5, CD51, CD52, CD56, CD6, CD74, CD80, CEA, CFD, CGRP, CLDN, CSFIR, CSF2, CTGF, CTLA-4, CXCR4, CXCR7, DKK1, DLL3, DLL4, DR5, EGFL7, EGFR, EPCAM, ERBB2, ERBB3, FAP, FGF23, FGFR1, GD2, GD3, GDF-8, GPNMB, GUCY2C, HER1, HER2, HGF, HIV-1, HSP90, ICAM-1, IFN-α, IFN-γ, IgE, CD221, IGF1, IGF2, IGHE, IL-1, IL2, IL-4, IL-5, IL-6, IL-6R, IL-9, IL-12 IL-15, IL-15R, IL-17, IL-13, IL-18, IL-1β, IL-22, IL-23, IL23A, integrins, ITGA2, IGTB2, Lewis-Y antigen, LFA-1, LOXL2, LTA, MCP-1, MIF, MS5A1, MUC1, MUC16, MSLN, myostatin, MMP superfamily, NCA-90, NFG, NOGO-A, Notch 1, NRP1, OX-40, OX-40L, P2X superfamily, PCSK9, PD-1, PD-L1, PDCD1, PDGF-R, RANKL, RHD, RON, TRN4, serum albumin, SDC1, SLAMF7, SIRPα, SOST, SHP1, SHP2, STEAP1, TAG-72, TEM1, TIGIT, TFPI, TGF-β, TNF-α, TNF superfamily, TRAIL superfamily, Toll-like receptors, WNT superfamily, VEGF-A, VEGFR-1, VWF, cytomegalovirus (CMV), respiratory syncytial virus (RSV), hepatitis B, hepatitis C, influenza A hemagglutinin, rabies virus, HIV virus, herpes simplex virus, and combinations thereof. Other targets or antigens can be found in U.S. Pat. Nos. 9,803,023, 9,663,582, and 20,170,349662, the contents of which are incorporated herein.

### III. Nucleic Acids

Another aspect of the invention features an isolated nucleic acid comprising a sequence that encodes the polypeptide or protein or antibody described above. A nucleic acid refers to a DNA molecule (e.g., a cDNA or genomic DNA), an RNA molecule (e.g., a mRNA), or a DNA or RNA analog. A DNA or RNA analog can be synthesized from nucleotide analogs. The nucleic acid molecule can be single-stranded or double-stranded, and preferably is double-stranded DNA. An “isolated nucleic acid” refers to a nucleic acid the structure of which is not identical to that of any naturally occurring nucleic acid or to that of any fragment of a naturally occurring genomic nucleic acid. The term, therefore, covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the polypeptide, fusion protein, or antibody of this invention. For this purpose, one can operatively link the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector includes a nucleic acid in a form suitable for expression of the nucleic acid in a host cell. Preferably the vector includes one or more regulatory sequences operatively linked to the nucleic acid sequence to be expressed.

A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce a polypeptide of this invention. A promoter is defined as a DNA sequence that directs RNA polymerase to bind to DNA and initiate RNA synthesis. A strong promoter is one which causes mRNAs to be initiated at high frequency.

Any polynucleotide as mentioned above or a biologically equivalent polynucleotide available to the artisan for the same intended purpose may be inserted into an appropriate expression vector and linked with other DNA molecules to form “recombinant DNA molecules” expressing this receptor. These vectors may be comprised of DNA or RNA; for most cloning purposes DNA vectors are preferred. Typical vectors include plasmids, modified viruses, bacteriophage and cosmids, yeast artificial chromosomes and other forms of episomal or integrated DNA. It is well within the purview of the artisan to determine an appropriate vector for a particular use.

A variety of mammalian expression vectors may be used to express the above-mentioned IgG Fcs in mammalian cells. As noted above, expression vectors can be DNA sequences that are required for the transcription of cloned DNA and the translation of their mRNAs in an appropriate host. Such vectors can be used to express eukaryotic DNA in a variety of hosts such as bacteria, blue-green algae, plant cells, insect cells, and animal cells. Specifically designed vectors allow the shuttling of DNA between hosts such as bacteria-yeast or bacteria-animal cells. An appropriately constructed expression vector should contain: an origin of replication for autonomous replication in host cells, selectable markers, a limited number of useful restriction enzyme sites, a potential for high copy number, and active promoters. Expression vectors may include, but are not limited to, cloning vectors, modified cloning vectors, specifically designed plasmids or viruses. Commercially available mammalian expression vectors which may be suitable, include but are not limited to, pcDNA3.neo (Invitrogen), pcDNA3.1 (Invitrogen), pCI-neo (Promega), pLITMUS28, pLITMUS29, pLITMUS38 and pLITMUS39 (New England Biolabs), pcDNAI, pcDNAIamp (Invitrogen), pcDNA3 (Invitrogen), pMClneo (Stratagene), pXT1 (Stratagene), pSG5 (Stratagene), EBO-pSV2-neo (ATCC 37593) pBPV-1 (8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565).

Also within the scope of this invention is a host cell that contains the above-described nucleic acid. Examples include bacterial cells (e.g., E. coli cells, insect cells (e.g., using baculovirus expression vectors), yeast cells, or mammalian cells. See, e.g., Goeddel, (1990) Gene Expression Technology: Methods in Enzymology 185, Academic Press, San Diego, Calif. To produce a polypeptide of this invention, one can culture a host cell in a medium under conditions permitting expression of the polypeptide encoded by a nucleic acid of this invention, and purify the polypeptide from the cultured cell or the medium of the cell. Alternatively, the nucleic acid of this invention can be transcribed and translated in vitro, e.g., using T7 promoter regulatory sequences and T7 polymerase.

All of naturally occurring IgG Fcs, genetic engineered IgG Fcs, and chemically synthesized IgG Fcs can be used to practice the invention disclosed therein. IgG Fc obtained by recombinant DNA technology may have the same amino acid sequence as SEQ ID NO: 2 or 3, or a functionally equivalent thereof. The term “IgG Fc” also covers chemically modified versions. Examples of chemically modified IgG Fc include IgG Fcs subjected to conformational change, addition or deletion of a sugar chain, and IgG Fc to which a compound such as polyethylene glycol has been bound.

One can verify the function and efficacy of a polypeptide/protein/antibody thus-made using an animal model as described below. Any statistically significant increase in in vivo half-life, increased affinity to an FcγR receptor (e.g., FcγRIIA, FcγRIIIA, or FcγRIIIB), FcRn, and/or enhanced cytotoxic activity indicates the polypeptide/protein/antibody is a candidate for treating the disorders mentioned below. The artisan will be capable of mixing and matching various research tools without undue experimentation. Once purified and tested by standard methods or according to the assays and methods described in the examples below, the polypeptide/protein/antibody can be included in the pharmaceutical composition for treating disorders as described below.

### IV. Compositions

Within the scope of this invention is a composition that contains a suitable carrier and one or more of the agents described above, such as the IgG Fc variant, related protein, or related antibody. The composition can be a pharmaceutical composition that contains a pharmaceutically acceptable carrier or a cosmetic composition that contains a cosmetically acceptable carrier.

The composition, in any of the forms described above, can be used for treating disorders described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono-or diglycerides). Fatty acid, such as, but not limited to, oleic acid and its glyceride derivatives, are useful in the preparation of injectables, as are natural pharmaceutically acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, TWEENS or SPANS or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally comprise one or more excipients or diluents. In some preferred embodiments, the topical formulations include a material that would enhance absorption or penetration of the active agent(s) through the skin or other affected areas. The topical composition is useful for treating inflammatory disorders in the skin, including, but not limited to eczema, acne, rosacea, psoriasis, contact dermatitis, and reactions to poison ivy.

A topical composition contains a safe and effective amount of a dermatologically acceptable carrier suitable for application to the skin. A “cosmetically acceptable” or “dermatologically-acceptable” composition or component refers a composition or component that is suitable for use in contact with human skin without undue toxicity, incompatibility, instability, allergic response, and the like. The carrier enables an active agent and optional component to be delivered to the skin at an appropriate concentration(s). The carrier thus can act as a diluent, dispersant, solvent, or the like to ensure that the active materials are applied to and distributed evenly over the selected target at an appropriate concentration. The carrier can be solid, semi-solid, or liquid. The carrier can be in the form of a lotion, a cream, or a gel, in particular, one that has a sufficient thickness or yield point to prevent the active materials from sedimenting. The carrier can be inert or possess dermatological benefits. It also should be physically and chemically compatible with the active components described herein, and should not unduly impair stability, efficacy, or other use benefits associated with the composition. The topical composition may be a cosmetic or dermatologic product in the form known in the art for topical or transdermal applications, including solutions, aerosols, creams, gels, patches, ointment, lotion, or foam.

### V. Treatment Methods

The agents described above can be administered to a subject for the prophylactic and therapeutic treatment various disorders, such as neoplastic disorders, inflammatory disorders, and infectious diseases. For example, the agents can be used in treating a viral or bacterial infection, a metabolic or autoimmune disorder, or cancer or other cellular proliferative disorder.

### A. Neoplastic Disorders

In one aspect, the present invention relates to the treatment of a subject in vivo using the above-described agents such that growth and/or metastasis of cancerous tumors is inhibited. In one embodiment, the invention provides a method of inhibiting growth and/or restricting the metastatic spread of tumor cells in a subject, comprising administering to the subject a therapeutically effective amount of an agent described above.

Non-limiting examples of preferred cancers for treatment include chronic or acute leukemia including acute myeloid leukemia, chronic myeloid leukemia, acute lymphoblastic leukemia, chronic lymphocytic leukemia, lymphocytic lymphoma, breast cancer, ovarian cancer, melanoma (e.g., metastatic malignant melanoma), renal cancer (e.g. clear cell carcinoma), prostate cancer (e.g. hormone-refractory prostate adenocarcinoma), colon cancer and lung cancer (e.g. non-small cell lung cancer). Additionally, the invention includes refractory or recurrent malignancies whose growth may be inhibited using the antibodies of the invention. Examples of other cancers that may be treated using the methods of the invention include bone cancer, pancreatic cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

The above treatment may also be combined with standard cancer treatments. For example, it may be effectively combined with chemotherapeutic regimes. In these instances, it may be possible to reduce the dose of chemotherapeutic reagent administered (Mokyr, M. et al. (1998) Cancer Research 58:5301-5304).

Other antibodies which may be used to activate host immune responsiveness can be used in combination with the agent of this invention. These include molecules targeting on the surface of dendritic cells which activate DC function and antigen presentation. For example, anti-CD40 antibodies are able to substitute effectively for T cell helper activity (Ridge, J. et al. (1998) Nature 393:474-478) and can be used in conjunction with the multi-specific molecule of this invention (Ito, N. et al. (2000) Immunobiology 201 (5) 527-40). Similarly, antibodies targeting T cell costimulatory molecules such as CTLA-4 (e.g., U.S. Pat. No. 5,811,097), CD28 (Haan, J. et al. (2014) Immunology Letters 162:103-112), OX-40 (Weinberg, A. et al. (2000) Immunol 164:2160-2169), 4-1BB (Melero, I. et al. (1997) Nature Medicine 3:682-685 (1997), and ICOS (Hutloff, A. et al. (1999) Nature 397:262-266) or antibodies targeting PD-1 (U.S. Pat. No. 8,008,449) PD-1L (U.S. Pat. Nos. 7,943,743 and 8,168,179) may also provide for increased levels of T cell activation. In another example, the multi-specific molecule of this invention can be used in conjunction with anti-neoplastic antibodies, such as RITUXAN (rituximab), HERCEPTIN (trastuzumab), BEXXAR (tositumomab), ZEVALIN (ibritumomab), CAMPATH (alemtuzumab), LYMPHOCIDE (epratuzumab), AVASTIN (bevacizumab), and TARCEVA (erlotinib), and the like.

### B. Inflammatory Disorder

The described invention provides methods for treating in a subject an inflammatory disorder. The term “inflammatory disorder” refers to a disorder that is characterized by abnormal or unwanted inflammation, such as an autoimmune disease. Autoimmune diseases are disorders characterized by the chronic activation of immune cells under non-activating conditions. Examples include psoriasis, inflammatory bowel diseases (e.g., Crohn's disease and ulcerative colitis), rheumatoid arthritis, psoriatic arthritis, multiple sclerosis, lupus, type I diabetes, primary biliary cirrhosis, and transplant.

Other examples of inflammatory disorders that can be treated by the methods of this invention include asthma, myocardial infarction, stroke, inflammatory dermatoses (e.g., dermatitis, eczema, atopic dermatitis, allergic contact dermatitis, urticaria, necrotizing vasculitis, cutaneous vasculitis, hypersensitivity vasculitis, eosinophilic myositis, polymyositis, dermatomyositis, and eosinophilic fasciitis), acute respiratory distress syndrome, fulminant hepatitis, hypersensitivity lung diseases (e.g., hypersensitivity pneumonitis, eosinophilic pneumonia, delayed-type hypersensitivity, interstitial lung disease (ILD), idiopathic pulmonary fibrosis, and ILD associated with rheumatoid arthritis), and allergic rhinitis. Additional examples also include myasthenia gravis, juvenile onset diabetes, glomerulonephritis, autoimmune thyroiditis, ankylosing spondylitis, systemic sclerosis, acute and chronic inflammatory diseases (e.g., systemic anaphylaxia or hypersensitivity responses, drug allergies, insect sting allergies, allograft rejection, and graft-versus-host disease), and Sjogren's syndrome.

A subject to be treated for an inflammatory disorder can be identified by standard diagnosing techniques for the disorder. Optionally, the subject can be examined for the level or percentage of one or more of cytokines or cells a test sample obtained from the subject by methods known in the art. If the level or percentage is at or below a threshold value (which can be obtained from a normal subject), the subject is a candidate for the treatment described herein. To confirm the inhibition or treatment, one can evaluate and/or verify the level or percentage of one or more of the above-mentioned cytokines or cells in the subject after treatment.

### C. Infectious Diseases

The present invention also relates to treating infectious diseases using the above-described agent that targets an antigen on or in a pathogen. Examples of infectious diseases herein include diseases caused by pathogens such as viruses, bacteria, fungi, protozoa, and parasites. Infectious diseases may be caused by viruses including adenovirus, cytomegalovirus, dengue, Epstein-Barr, hanta, hepatitis A, hepatitis B, hepatitis C, herpes simplex type I, herpes simplex type II, human immunodeficiency virus, (HIV), human papilloma virus (HPV), influenza, measles, mumps, papova virus, polio, respiratory syncytial virus, rinderpest, rhinovirus, rotavirus, rubella, SARS virus, smallpox, viral meningitis, and the like. Infectious diseases may also be caused by bacteria including Bacillus antracis, Borrelia burgdorferi, Campylobacter jejuni, Chlamydia trachomatis, Clostridium botulinum, Clostridium tetani, Diptheria, E. coli, Legionella, Helicobacter pylori, Mycobacterium rickettsia, Mycoplasma nesisseria, Pertussis, Pseudomonas aeruginosa, S. pneumonia, Streptococcus, Staphylococcus, Vibrio cholera, Yersinia pestis, and the like. Infectious diseases may also be caused by fungi such as Aspergillus fumigatus, Blastomyces dermatitidis, Candida albicans, Coccidioides immitis, Cryptococcus neoformans, Histoplasma capsulatum, Penicillium marneffei, and the like. Infectious diseases may also be caused by protozoa and parasites such as chlamydia, kokzidioa, Leishmania, malaria, rickettsia, Trypanosoma, and the like.

The treatment method can be performed in vivo or ex vivo, alone or in conjunction with other drugs or therapy. A therapeutically effective amount can be administered in one or more administrations, applications or dosages and is not intended to be limited to a particular formulation or administration route.

The agent can be administered in vivo or ex vivo, alone or co-administered in conjunction with other drugs or therapy, i.e., a cocktail therapy. As used herein, the term “co-administration” or “co-administered” refers to the administration of at least two agents or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In an in vivo approach, a compound or agent is administered to a subject. Generally, the compound or agent is suspended in a pharmaceutically-acceptable carrier (such as, for example, but not limited to, physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Variations in the needed dosage are to be expected in view of the variety of compounds/agents available and the different efficiencies of various routes of administration. For example, oral administration would be expected to require higher dosages than administration by i.v. injection. Variations in these dosage levels can be adjusted using standard empirical routines for optimization as is well understood in the art.

Encapsulation of the compound in a suitable delivery vehicle (e.g., polymeric microparticles or implantable devices) can increase the efficiency of delivery, particularly for oral delivery.

### VI. EXAMPLES

### Example 1

This example describes the material and methods used in Examples 2-3 bellow

### Materials and Methods

### Mouse Strains

All mouse in vivo experiments were performed in compliance with federal laws and institutional guidelines and had been approved by the Rockefeller University Institutional Animal Care and Use Committee. Mice were bred and maintained at the Comparative Bioscience Center at the Rockefeller University. The following strains were used for experiments: (i) FcγR deficient mice (FcγR^{null}), previously developed and characterized in Smith, P et al. Proc Natl Acad Sci U S A 109, 6181-6186 (2012); (ii) FcγR humanized mice (mFcγRα^{null}, Fcgr1^{-/-}, hFCGR1A⁺, hFCGR2A⁺, hFCGR2B⁺, hFCGR3A⁺, hFCGR3B⁺) generated and extensively characterized in Smith, P et al. Proc Natl Acad Sci U S A 109, 6181-6186 (2012); (iii) FcγR/FcRn humanized mice (mFcγRα^{null}, Fcgr1^{-/-}, Fcgrt^{-/-}, hFCGR1A⁺, hFCGR2A⁺, hFCGR2B⁺, hFCGR3A⁺, hFCGR3B⁺, hFCGRT⁺) were generated by crossing FcγR humanized mice with FcRn humanized mice (developed in Petkova, S. B. et al. Int Immunol 18, 1759-1769); (iv) FcγR/CD20 humanized mice (mFcγRα^{null}, Fcgr1^{-/-}, hFCGR1A⁺, hFCGR2A⁺, hFCGR2B⁺, hFCGR3A⁺, hFCGR3B⁺, hCD20⁺).

### Surface Plasmon Resonance (SPR) Analysis

FcγR and FcRn binding affinity of the human IgG1 Fc domain variants was determined by surface plasmon resonance (SPR), using previously described protocols (Wang, T. T. et al. Science 355, 395-398 (2017) and Li, T. et al. Proc Natl Acad Sci U S A 114, 3485-3490, (2017)). All experiments were performed on a Biacore T200 SPR system (GE Healthcare) at 25° C. in HBS-EP⁺ buffer (pH 7.4 for FcγRs, pH 6.0 for FcRn). Recombinant protein G (Thermo Fisher) was immobilized to the surface of CM5 sensor chip (GE Healthcare) using amine coupling chemistry at a density of 500 resonance units (RU). Human IgG1 Fc variants were captured on the Protein G-coupled surface (250 nM injected for 60 s at 20 μl/min) and recombinant human, rhesus, or mouse FcγR ectodomains (7.8125-2000 nM; Sino Biological) or human FcRn/β2 microglobulin (1.95-500 nM; Sino Biological) were injected through flow cells at a flow rate of 20 μl/min. Association time was 60 s followed by a 600-s dissociation step. At the end of each cycle, the sensor surface was regenerated with 10 mM glycine, pH 2.0 (50 μl/min; 40 s). Background binding to blank immobilized flow cells was subtracted, and affinity constants were calculated using BIAcore T200 evaluation software (GE Healthcare) using the 1:1 Langmuir binding model.

### In Vivo Cytotoxicity Models

Platelet, CD4⁺ T cell-, and hCD20⁺ B-cell depletion experiments were performed in FcγR humanized and FcγR/FcRn humanized mice using previously described protocols (Smith, P et al. Proc Natl Acad Sci U S A 109, 6181-6186 (2012) and Wang, T. T. et al. Science 355, 395-398 (2017). Rhesus B-cell depletion experiments involved the administration (i.v.) of wild-type human IgG1 or GAALIE (G236A/A330L/1332E) variants of the anti-CD20 mAb 2B8 to rhesus monkeys (i.v.) at 0.05 mg/kg. CD20⁺ frequencies and cell numbers were analyzed in blood by flow cytometry at various time points before and after antibody administration.

### Antibody Expression, Purification, and Analysis

Antibodies were generated by transient transfection of HEK293T or Expi293 cells, as previously described in Bournazos, S. et al. Cell 158, 1243-1253 (2014). Antibodies were purified using Protein G Sepharose 4 Fast Flow or MabSelect SuRe LX affinity purification media (GE Healthcare). Purified proteins were dialyzed in PBS and sterile filtered (0.22 μm). Purity was assessed by SDS-PAGE and Coomassie staining and was estimated to be >90%. Protein Tm was determined using the Protein Thermal Shift Dye Kit (ThermoFisher) following manufacturer's instructions on a QuantStudio 6K Flex real-time thermal cycler.

### Quantification of Serum IgG Levels

For the quantitation of serum concentration of human IgG1 variants, neutravidin-coated plates were used (5 μg/ml; overnight). Plates were incubated with either biotinylated goat anti-human IgG (mouse IgG absorbed, Jackson Immunoresearch) for mouse serum samples, or CaptureSelect™ Human IgG-Fc PK Biotin Conjugate for rhesus plasma samples. Following incubation (60 min at room temperature), plates were blocked with PBS+2% (w/v) BSA+0.05% (v/v) Tween20 for 2 h. Serially diluted (1:3 starting with an initial 1:10 dilution) serum samples were incubated for 1 h. IgG binding was detected using goat anti-human IgG (Fcγ-specific, 1 h; 1:5000; Jackson Immunoresearch). Plates were developed using the TMB (3,3′,5,5′-Tetramethylbenzidine) two-component peroxidase substrate kit (KPL) and reactions stopped with the addition of 1 M phosphoric acid. Absorbance at 450 nm was immediately recorded using a SpectraMax Plus spectrophotometer (Molecular Devices), and background absorbance from negative control samples was subtracted.

### Example 2

An Fc domain variant (termed GASDALIE), which encompasses specific mutations (G236A/S239D/A330L/1332E) at the amino acid backbone of human IgG1, was developed. It exhibits selectively enhanced binding to the activating human FcγRs, FcγRIIa and FcγRIIIa (Smith, P., DiLillo, D. J., Bournazos, S., Li, F. & Ravetch, J. V. Mouse model recapitulating human Fcgamma receptor structural and functional diversity. Proc Natl Acad Sci U S A 109, 6181-6186 (2012)). In diverse models of antibody-mediated protection against bacterial and viral infection, the GASDALIE Fc domain variant of protective mAbs demonstrated significantly enhanced protective activity compared to wild-type human IgG1. See, Smith, P., et al. Proc Natl Acad Sci U S A 109, 6181-6186 (2012); Bournazos, S. et al. Cell 158, 1243-1253 (2014); Bournazos, S., et al. J Clin Invest 124, 725-729 (2014); and DiLillo, D. J., et al. Nat Med 20, 143-151 (2014).

More importantly, evaluation of the therapeutic activity of GASDALIE variant of anti-CD20 mAbs in a mouse model of CD20+ lymphoma revealed that this variant exhibited not only improved cytotoxic activity against CD20+ lymphoma cells, but also stimulated the induction of long-term T-cell memory responses, which conferred protection against subsequent lymphoma challenge (DiLillo, D. J. et al. Cell 161, 1035-1045 (2015)). Mechanistic studies revealed that whereas enhanced cytotoxicity during the primary lymphoma challenge was mediated through enhanced engagement of FcγRIIIa on effector leukocytes, like monocytes and macrophages, crosslinking of FcγRIIa on dendritic cells promoted dendritic cell maturation and the induction of T-cell memory responses that mediated protection upon secondary challenge (DiLillo, D. J. et al. Cell 161, 1035-1045 (2015)). Collectively, these studies demonstrated improved therapeutic activity for the GASDALIE Fc domain variant that is accomplished through selectively augmented binding to human FcγRIIa and FcγRIIIa.

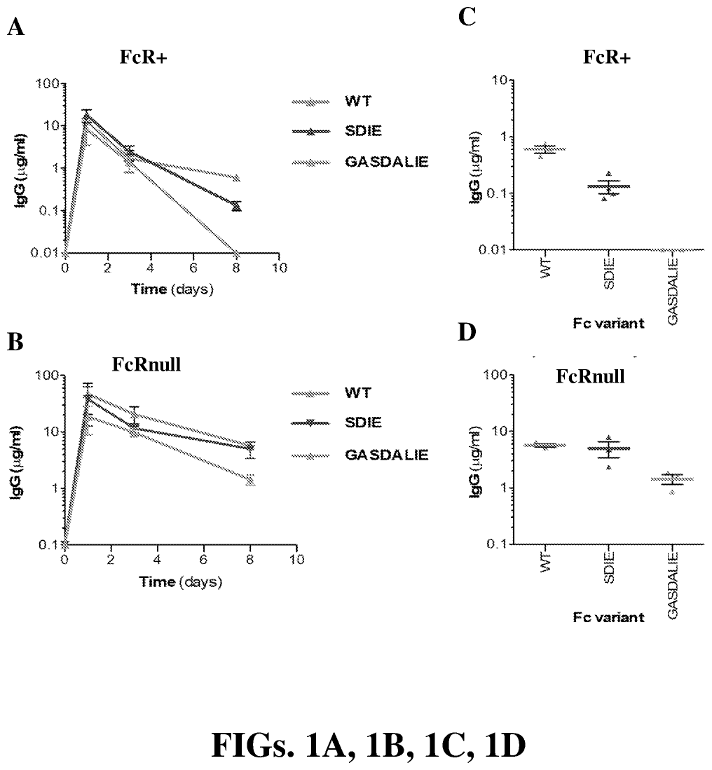

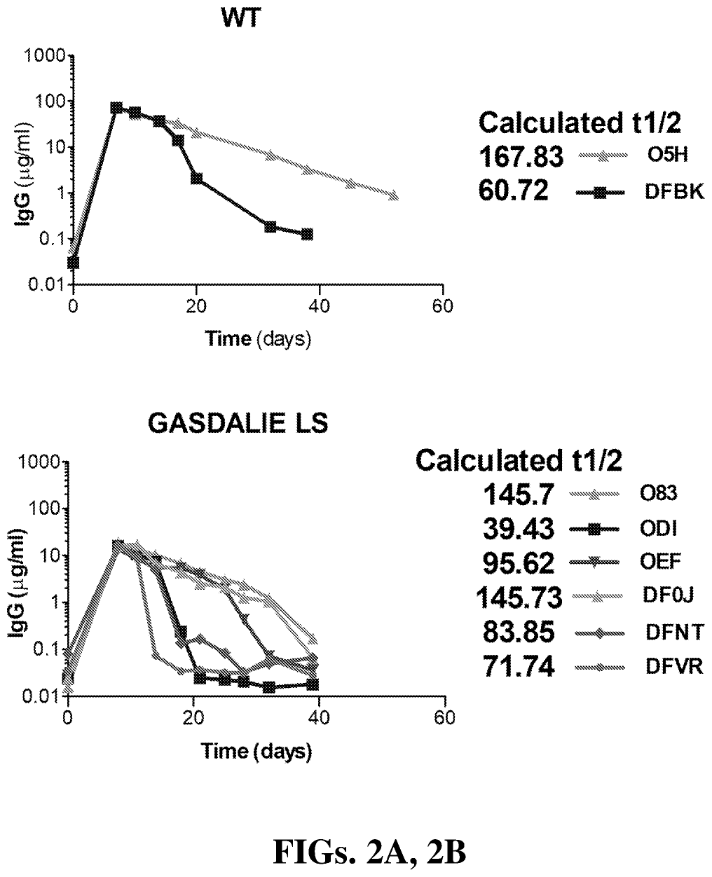

Despite its improved Fc effector function, the GASDALIE variant exhibited significantly shorter half-life in vivo primarily in FcγR humanized mice and to a lesser extent in mouse strains deficient for all classes of FcγRs (FIG. 1). This effect could be attributed to its increased affinity for FcγRs, as well as to decreased in vivo protein stability. Even when combined with Fc domain mutations (e.g., LS: M428L/N434S) that increase FcRn affinity and extend half-life, the GASDALIE Fc domain variant exhibited very short half-life in vivo in non-human primates (FIG. 2).

The inventors developed an Fc domain variant (termed GAALIE) that exhibits all the characteristics of the GASDALIE variant, including increased FcγRIIa and FcγRIIIa affinity and enhanced cytotoxic activity in several mAb-mediated cytotoxicity models, but unexpectedly it also maintains physiological half-life. In the studies shown below, inventors included Fc domain variants (afucosylated and the S239D/1332E variant) that have already been evaluated in humans and exhibit increased FcγR binding affinity without significant impairment in their in vivo stability and half-life. Goede, V. et al. N Engl J Med 370, 1101-1110 (2014); Zalevsky, J. et al. Blood 113, 3735-3743 (2009); and Woyach, J. A. et al. Blood 124, 3553-3560 (2014).

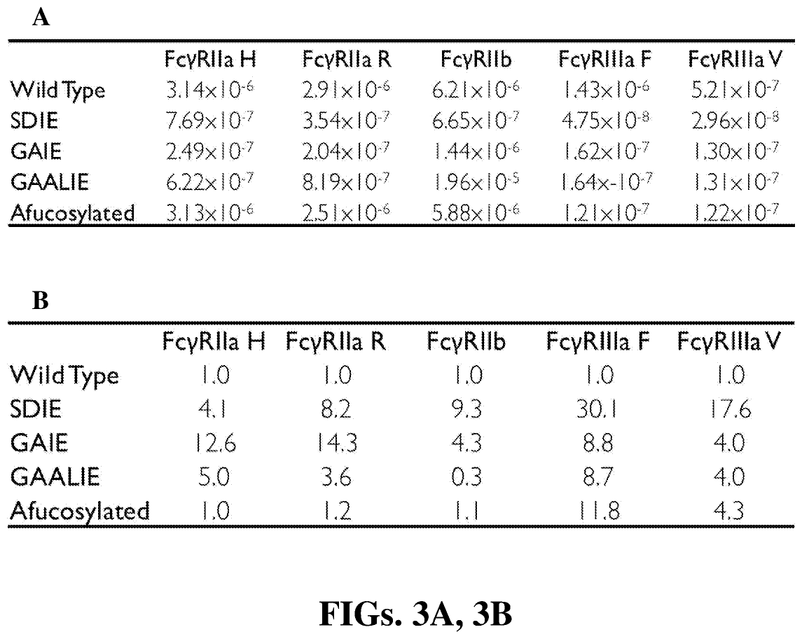

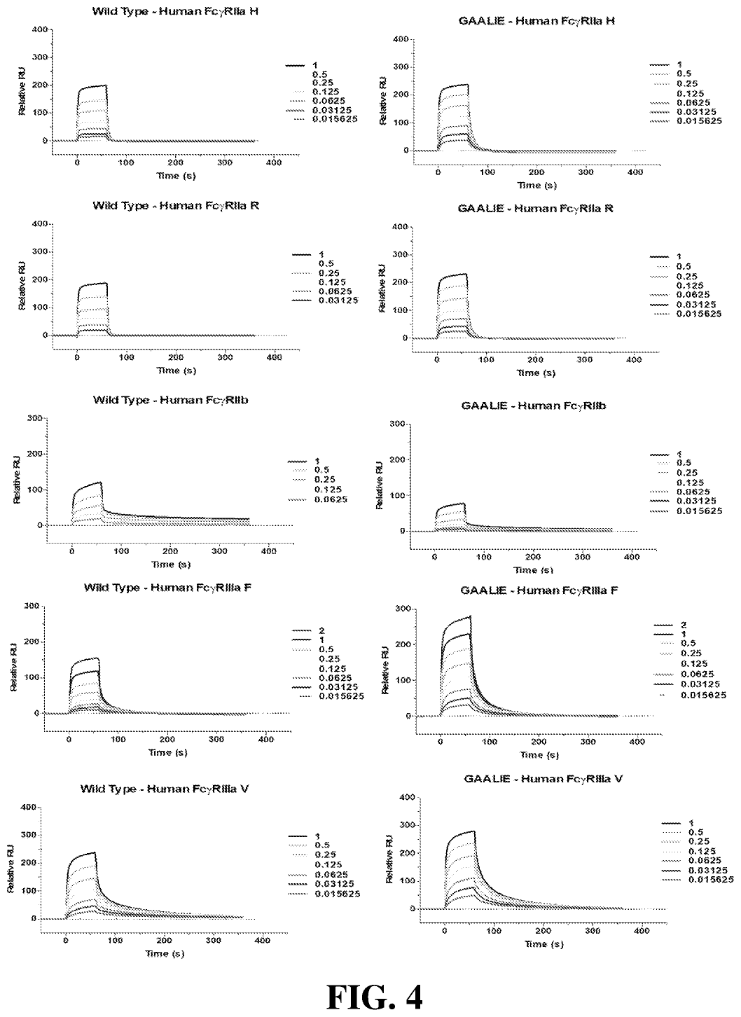

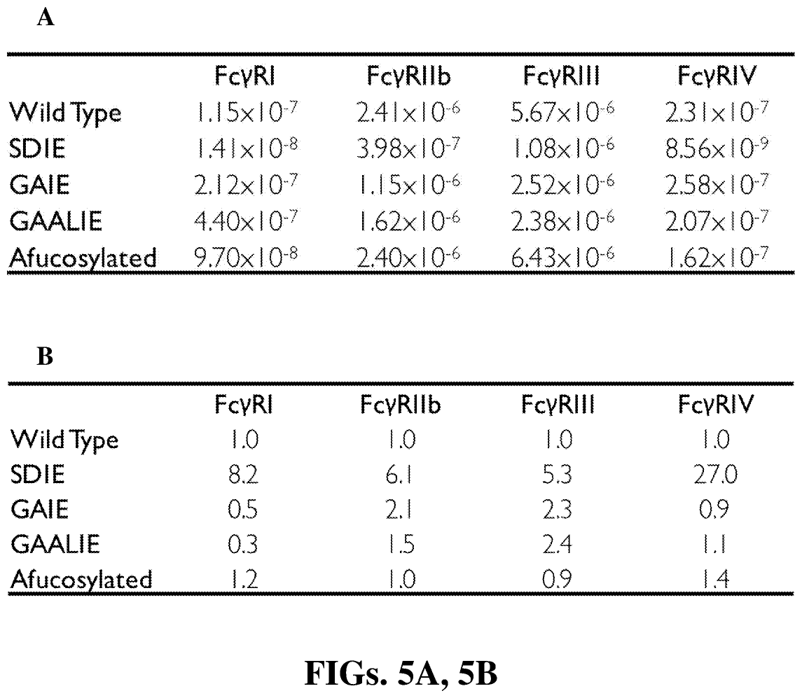

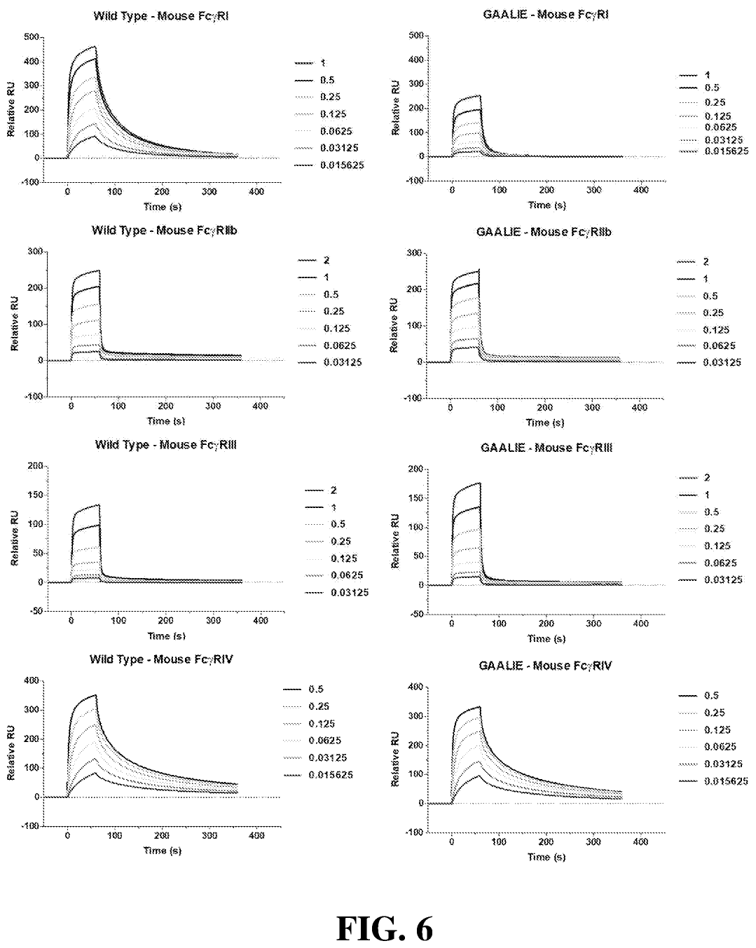

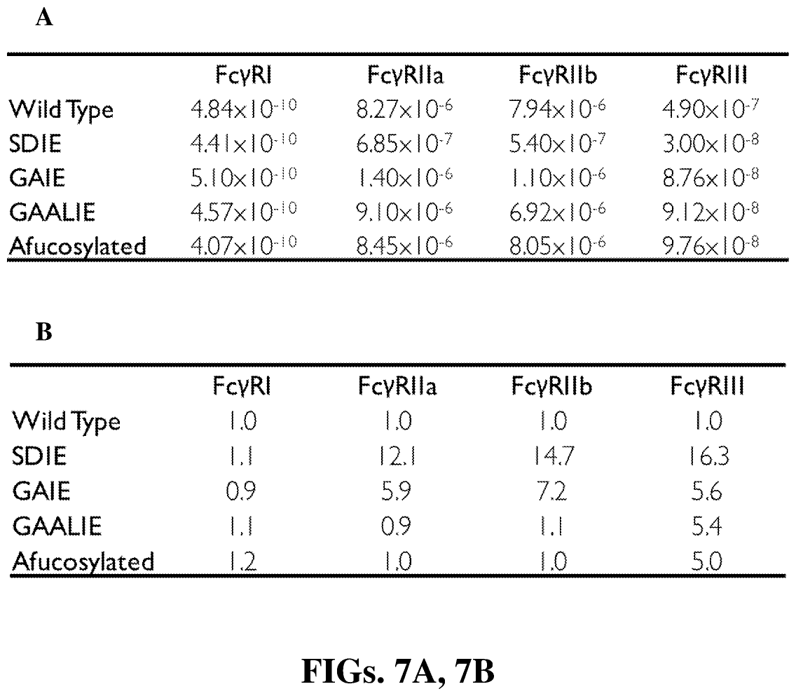

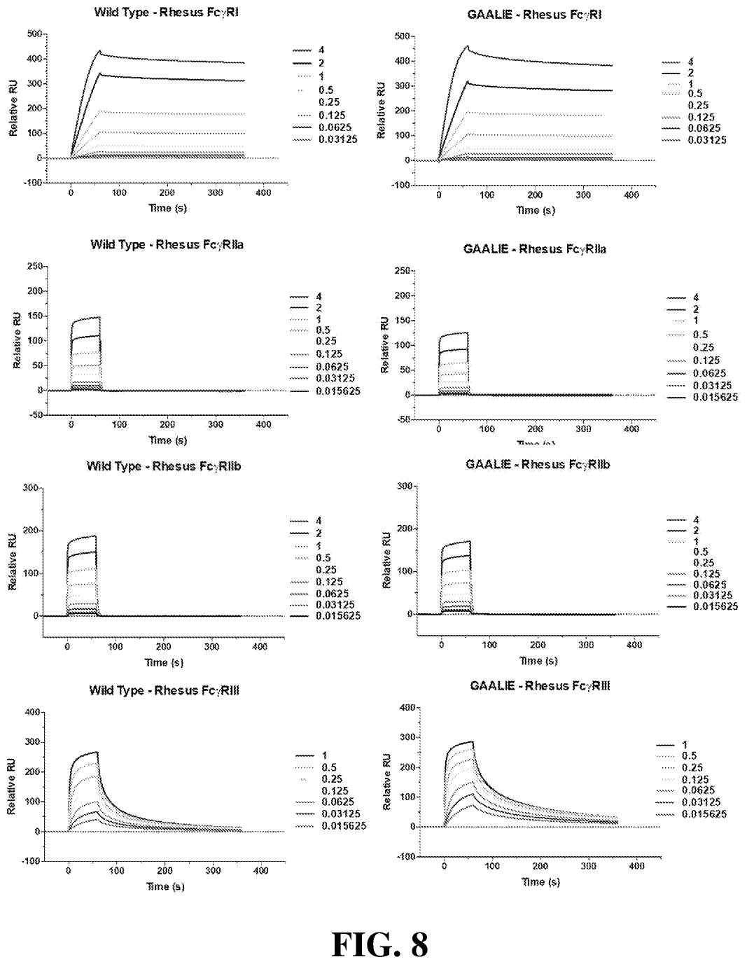

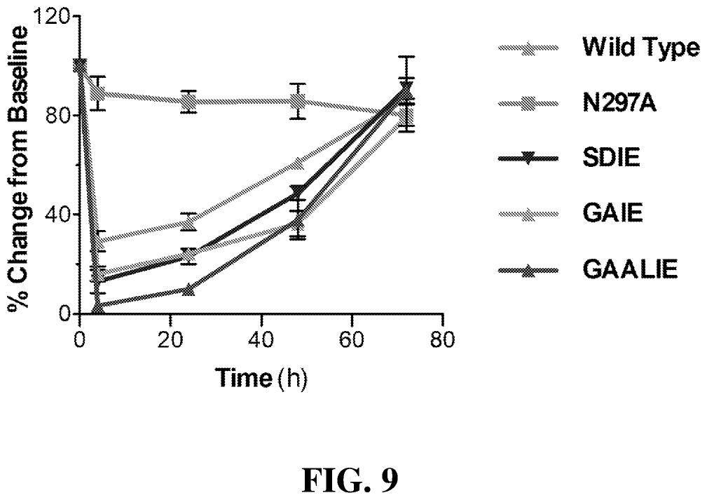

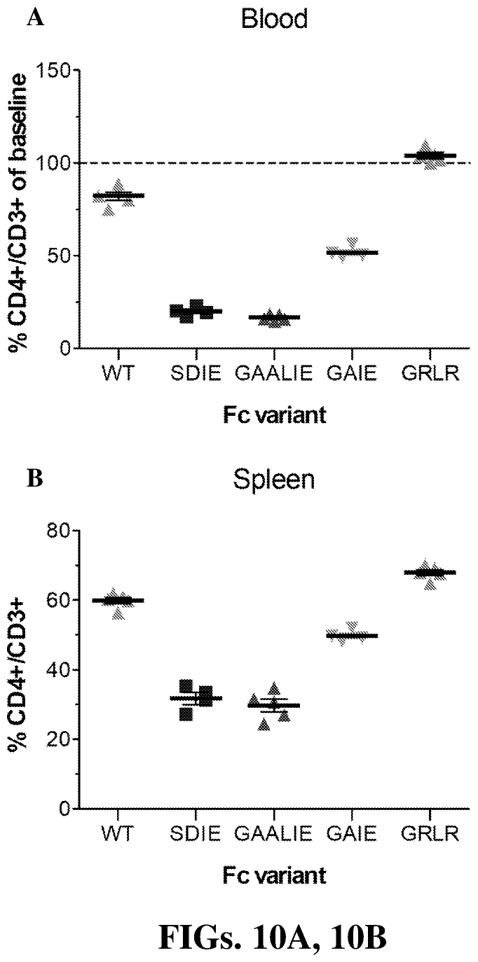

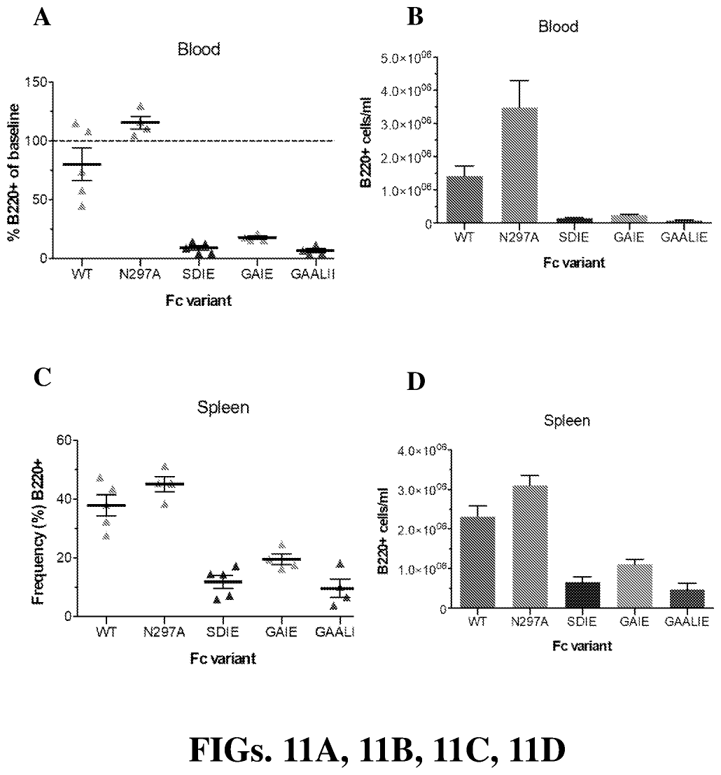

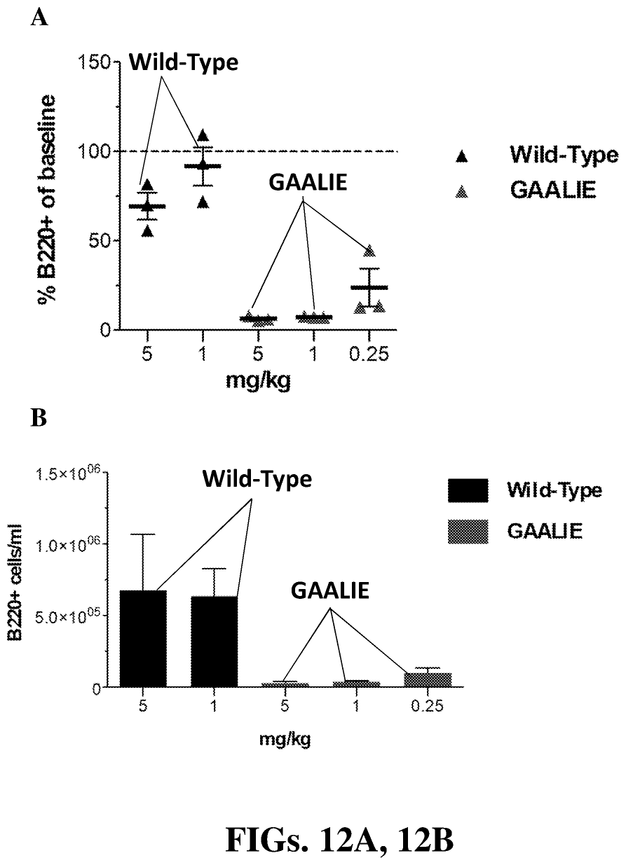

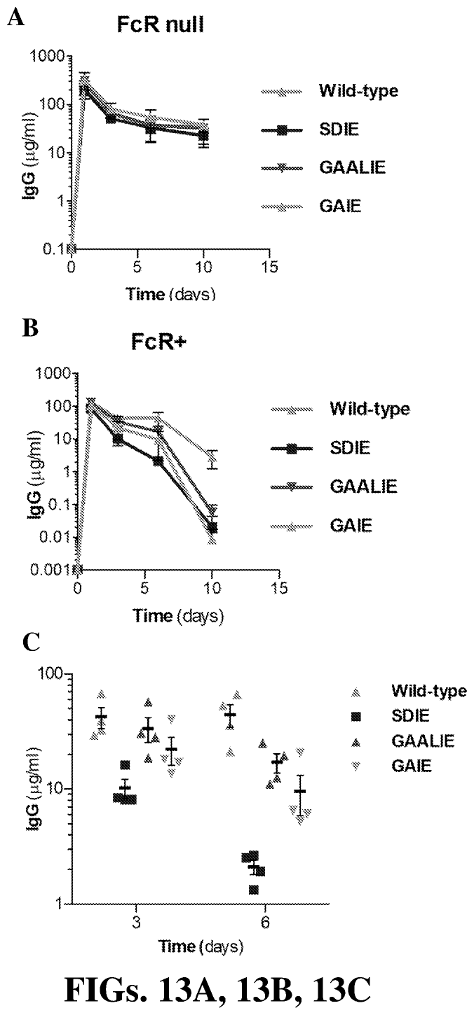

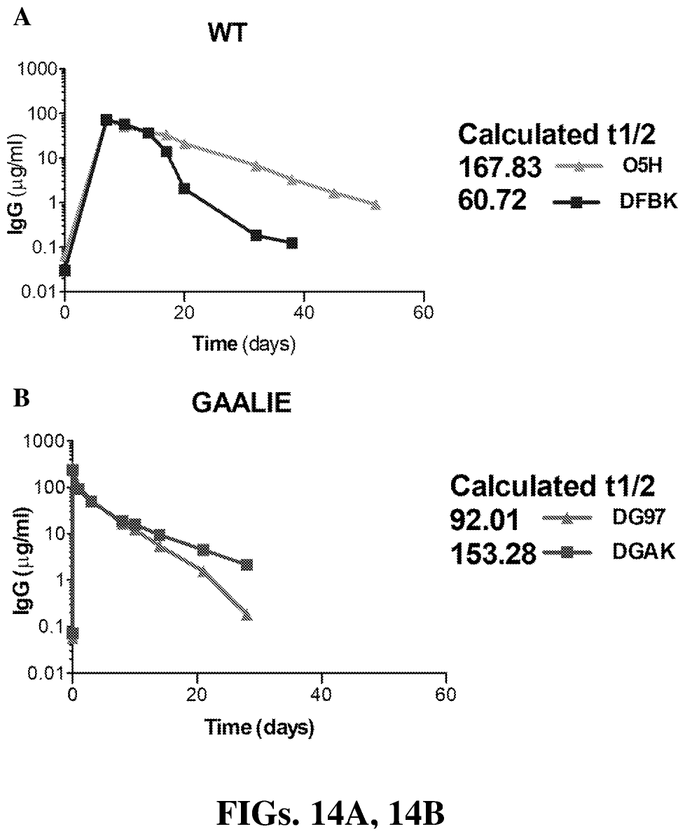

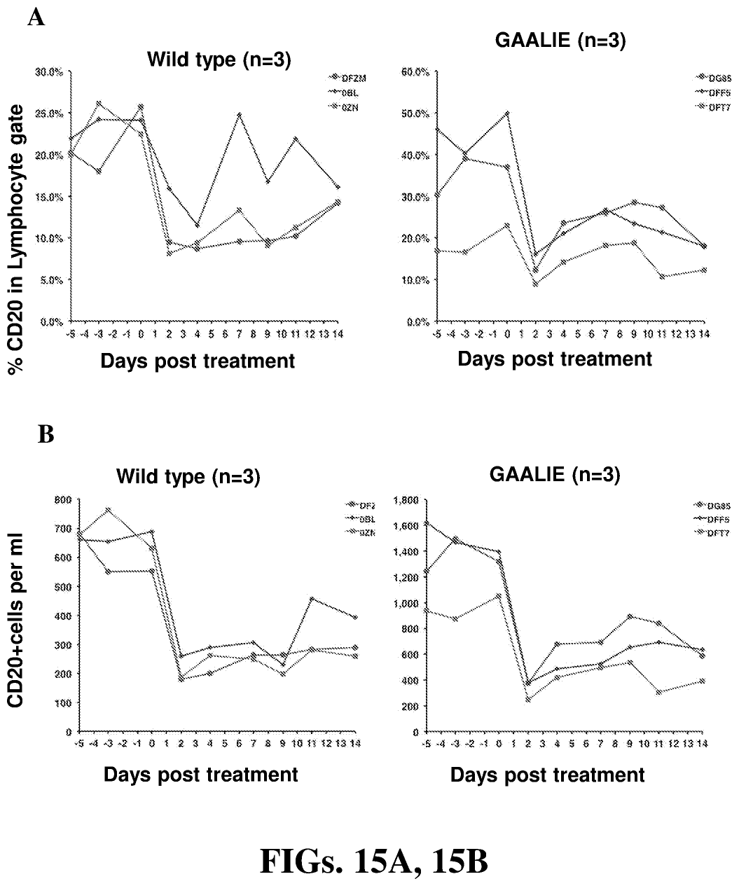

The GAALIE variant (G236A/A330L/1332E) was characterized for its affinity for all classes of human, rhesus, and mouse FcγRs (FIGS. 3-8), as well as for its cytotoxic effector activity in platelet, CD4+ T-cell, and B-cell depletion models in FcγR humanized mice (FIGS. 9-12). Evaluation of the half-life of the GAALIE variant in FcγR humanized and FcγR-deficient mice, as well as in rhesus monkeys revealed that it exhibited physiological half-life (FIGS. 13-14). Additionally, the in vivo cytotoxic of the GAALIE variant was assessed in non-human primates (Rhesus macaques) in a model of mAb-mediated depletion of CD20+ B cells (FIG. 15).

### Example 3

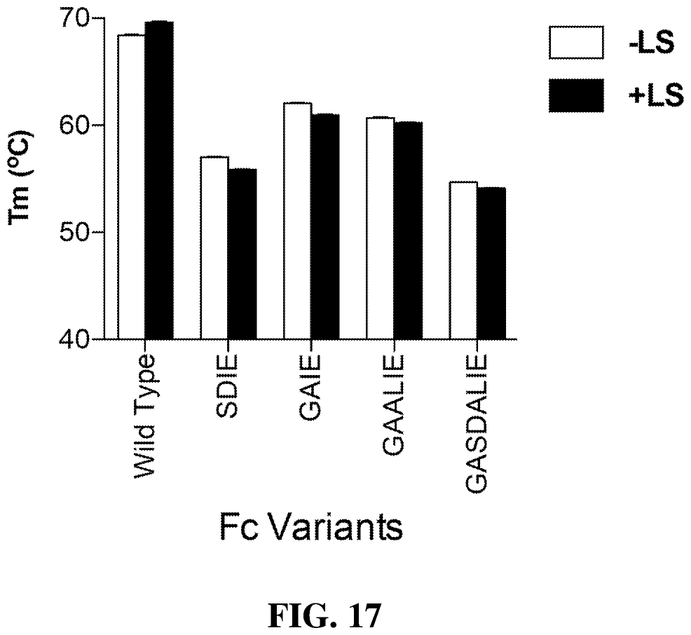

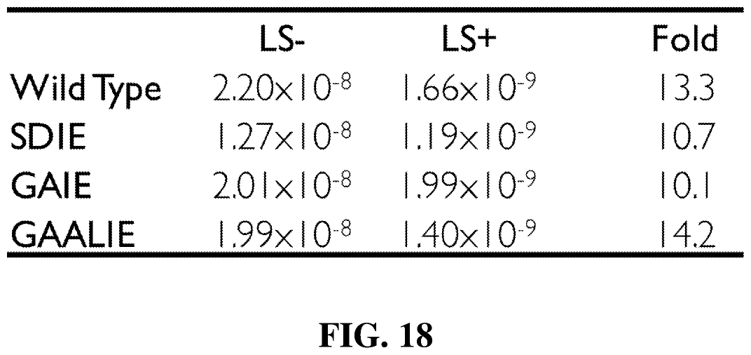

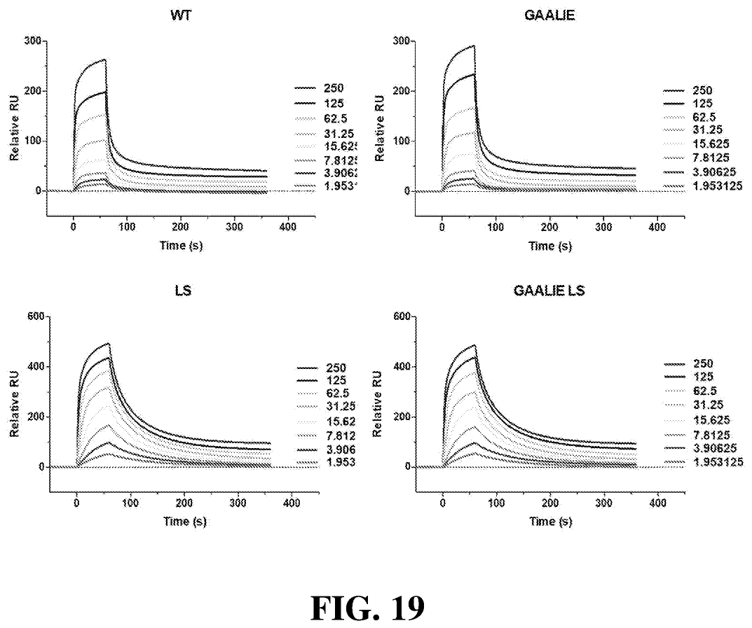

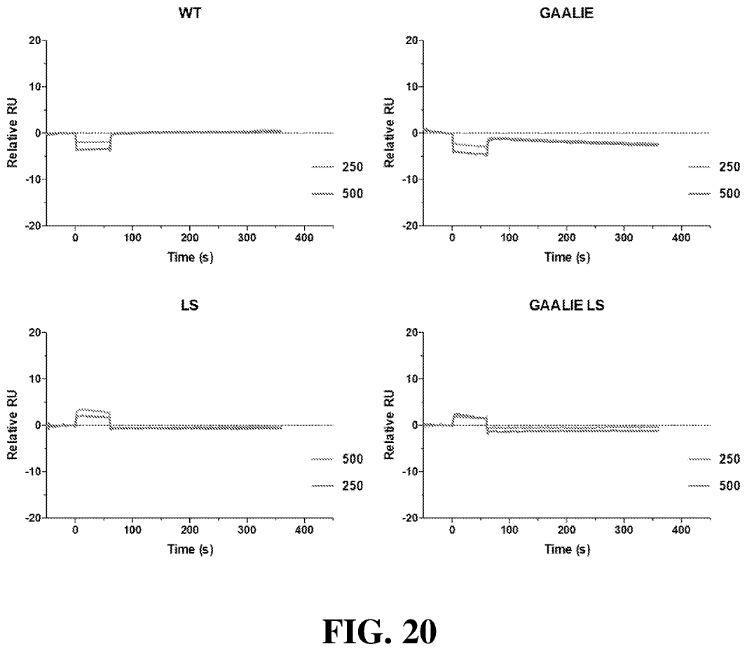

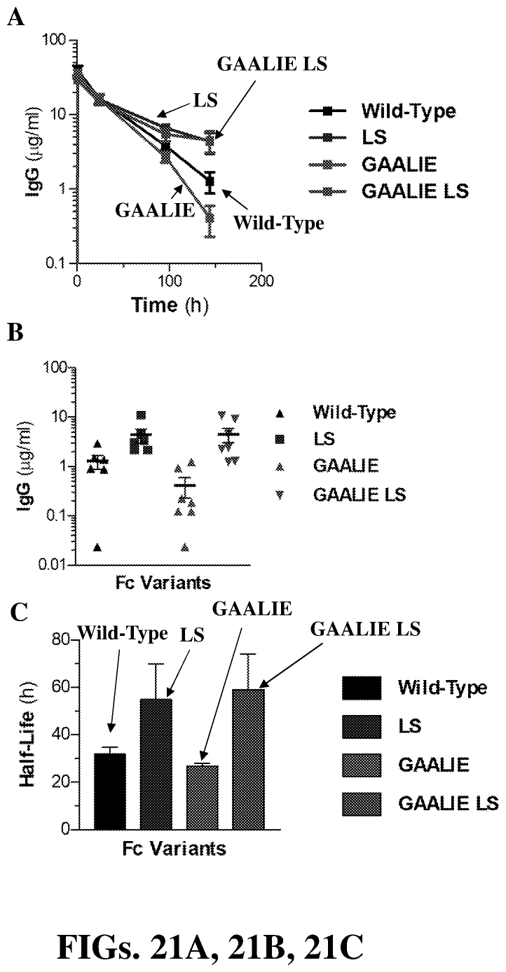

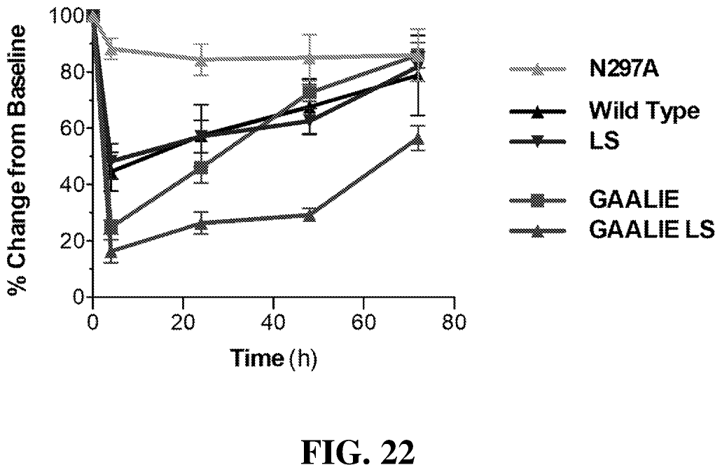

To further extend the in vivo half-life of the GAALIE variant, it was combined with mutations that increase FcRn affinity without impacting FcγR binding (Zalevsky, J. et al. Nat Biotechnol 28, 157-159 (2010) and Grevys, A. et al. J Immunol 194, 5497-5508 (2015)). These mutations include M428L and N434S (LS variant, Zalevsky, J. et al. Nat Biotechnol 28, 157-159 (2010)) and the amino acid sequence of the generated Fc domain variants is presented in FIG. 16. Protein melting temperature and FcRn binding affinity of the FcγR/FcRn-enhancing variants was determined (FIGS. 17-20). Additionally, the in vivo half-life of these variants was evaluated in FcRn/FcγR humanized mice (FIG. 21). As expected, GAALIE LS (G236A/A330L/1332E/M428L/N434S) exhibited extended half-life, which also translated to prolonged and enhanced Fc effector activity in a model of mAb-mediated platelet depletion in FcγR/FcRn humanized mice (FIG. 22).

### Example 4

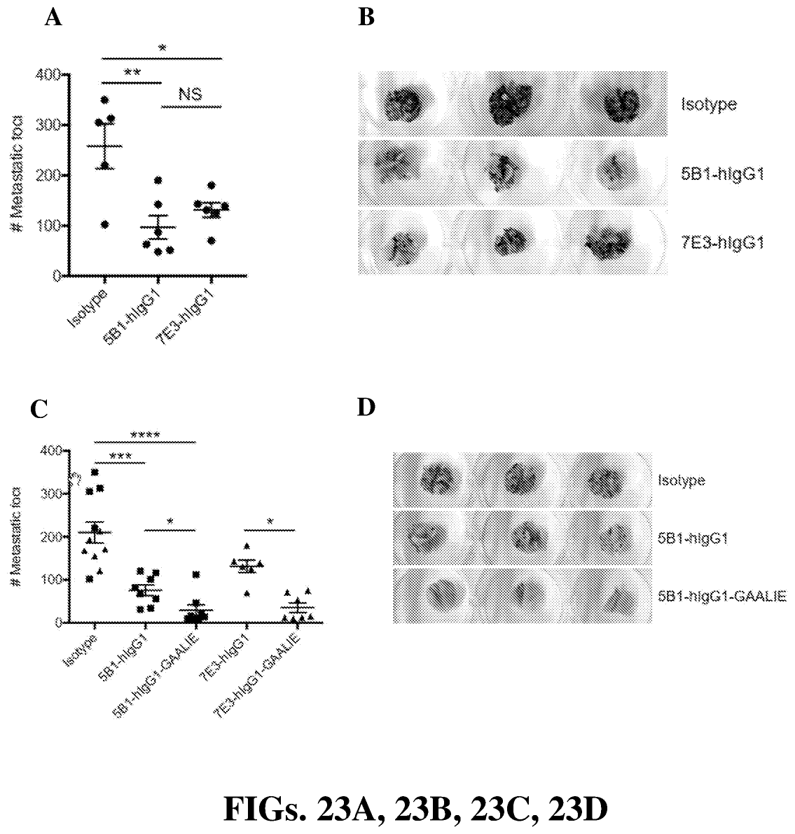

In order to recapitulate the interactions of antibodies designed for clinical use with a human Fc with human FcRs, B16-FUT3 cells were inoculated to FcγR-humanized mice, a strain which lacks all murine FcRs while carrying transgenes of all human FcγRs (Smith, P., et al. Proc Natl Acad Sci U S A 109, 6181-6186 (2012)), resulting in the recapitulation of the cellular expression pattern of human FcRs in a fully immunocompetent murine background. B16 tumor-bearing mice were treated with sLeA-targeting antibodies, clones 5B1 and 7E3, expressing the hIgG1 subclass. Both 5B1 and 7E3 clones exhibited comparable therapeutic efficacy (FIG. 23A), leading to a significant reduction in the number of metastatic foci in the lungs. As observed with the chimeric human-mouse antibodies (data not shown), engineering 5B1-hIgG1 with an Fc mutation (N297A) that abolishes its ability engage human FcRs results in the loss of the therapeutic effect of sLeA-targeting antibodies (data not shown).

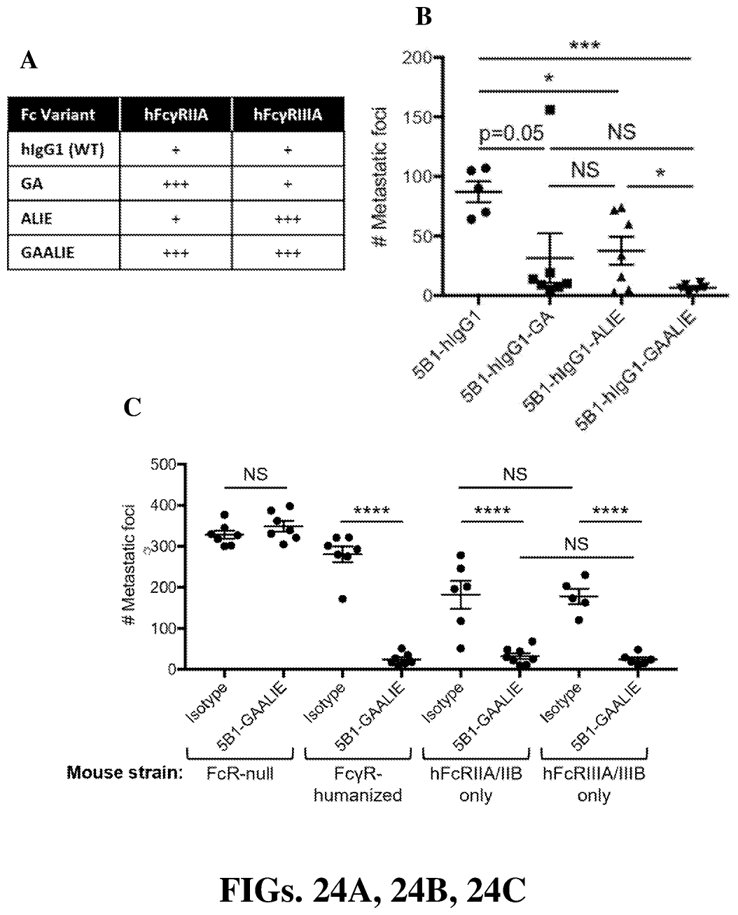

In light of the above-described role of activating FcRs in mediating antibody-induced tumor clearance, it was sought to increase the therapeutic potency of sLeA-targeting antibodies by increasing their affinity to activating FcRs. In doing so, hIgG1 sLeA-targeting antibodies were re-engineered by introducing three point mutations (G236A/A330L/1332E) (“GAALIE”). The GAALIE point mutations significantly enhanced the affinity of sLeA-targeting antibodies to two activating human FcRs: hFcγRIIA and hFcγRIIIA while reducing the binding to the inhibitory receptor, hFcRIIB, without interfering with their binding affinity towards sLeA. The re-engineered 5B1 and 7E3 antibody variants demonstrated superior anti-tumor activity compared to the parental antibody with a wild-type hIgG1 Fc portion (FIG. 24B). These findings reinforce the findings that engagement of activating FcRs is a crucial step in the process of efficient antibody-mediated tumor clearance.

### Example 5

The engagement of hFcγRIIIA alone is both necessary and sufficient for antibody-mediated tumor clearance in several tumor models, while the engagement of the activating receptor hFcγRIIA was insufficient to mediate tumor clearance. In this study, it was aimed to determine whether these findings also hold true for carbohydrate-targeting antibodies. The anti-tumor activity of three Fc variants with enhanced affinities to either hFcγRIIA (GA), hFcγRIIIA (ALIE) or both (GAALIE) in FcγR-humanized tumor-bearing mice were compared (FIG. 24A). The affinity of the GA and ALIE hIgG1 Fc variants to different human FcRs has been reported9,34,35; the GAALIE Fc variant exhibits a higher affinity to hFcRIIA and hFcRIIIA, with reduced affinity to hFcRIIB, and an in vivo half-life comparable to hIgG1, while demonstrating a superior ADCC capability compared to the parental hIgG1 (data not shown).

All three Fc variants exhibited a comparable anti-tumor potential, which was significantly higher than that of the wild-type parental human IgG1 antibody (FIG. 24B). To confirm these findings, the anti-tumor activity of the Fc variant 5B1-hIgG1-GAALIE (with enhanced affinity to both activating FcRs) in several transgenic mouse strains expressing human FcRs were compared. FIG. 24C indicates that the 5B1-hIgG1-GAALIE variant demonstrates a pronounced, yet comparable, anti-tumor activity not only in FcγR-humanized mice (which express all human FcγRs, including hFcγRIIA, hFcγRIIB, and hFcγRIIIA), but also in hFcγRIIA-only mice and hFcγRIIIA-only mice. As expected, tumor clearance was not observed in FcR-null mice. NK depletion did not substantially hamper the anti-tumor activity of this sLeA-targeting antibody (data not shown), suggesting that tumor cell depletion is primarily mediated by effector cells expressing hFcγRIIIA and hFcRγIIA, such as macrophages.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed is:

1. A method of treating a patient by administering an anti-hepatitis, anti-influenza, anti-SARS, anti-HIV, anti-CTLA-4, anti-CCR8, anti-sLeA, anti-CD47, anti-EGFR, anti-CD20, or anti-CD4 antibody comprising:
a) means for binding human hepatitis, influenza, SARS, HIV, CTLA-4, CCR8, sLeA, CD47, EGFR, CD20, or CD4 protein; and
b) an Fc variant comprising an Alanine (A) at position 236, a Leucine (L) at position 330, and a Glutamic acid (E) at position 332, wherein the numbering is according to the EU index, and wherein an antibody having the Fc variant has a higher binding affinity to hFcRIIA and FcRIIIA and reduced binding affinity to FcRIIB compared to the antibody having the wild-type IgG1 Fc sequence of SEQ ID NO: 1.

2. The method of claim 1, wherein the Fc variant further comprises a Leucine (L) at position 428, and a Serine(S) at position 434.

3. The method of claim 1, wherein the Fc variant does not comprise an Aspartic acid (D) at position 239.

4. The method of claim 1, wherein the Fc variant comprises the sequence of SEQ ID NO: 2 or 3.

5. The method of claim 1, wherein the antibody is selected from the group consisting of a chimeric antibody, a humanized antibody, and a human antibody.

6. The method of claim 2, wherein the antibody has an extended half-life as compared to an antibody having the sequence of SEQ ID NO: 1.

7. The method of claim 1, wherein the patient has an inflammatory disorder, a neoplastic disorder, or an infectious disease.

8. The method of claim 6, wherein the patient has an inflammatory disorder.

9. The method of claim 6, wherein the patient has a neoplastic disorder.

10. The method of claim 6, wherein the patient has an infectious disease.

11. A method of treating a patient by administering an anti-hepatitis, anti-influenza, anti-SARS, anti-HIV, anti-CTLA-4, anti-CCR8, anti-sLeA, anti-CD47, anti-EGFR, anti-CD20, or anti-CD4 antibody comprising:
a) means for binding human hepatitis, influenza, SARS, HIV, CTLA-4, CCR8, sLeA, CD47, EGFR, CD20, or CD4 protein; and
b) an Fc variant comprising an Alanine (A) at position 236, a Leucine (L) at position 330, a Glutamic acid (E) at position 332, a Leucine (L) at position 428, and a Serine(S) at position 434, wherein the numbering is according to the EU index, and wherein an antibody having the Fc variant has an extended half life as compared to the antibody having the wild-type IgG1 Fc sequence of SEQ ID NO: 1.

12. The method of claim 11, wherein the Fc variant comprises the sequence of SEQ ID NO: 3.

13. The method of claim 11, wherein the antibody is selected from the group consisting of a chimeric antibody, a humanized antibody, and a human antibody.

14. The method of claim 11, wherein the patient has an inflammatory disorder, a neoplastic disorder, or an infectious disease.

15. The method of claim 13, wherein the patient has an inflammatory disorder.

16. The method of claim 13, wherein the patient has a neoplastic disorder.

17. The method of claim 13, wherein the patient has an infectious disease.

18. The method of claim 13, wherein the antibody has a higher binding affinity to hFcRIIA and FcRIIIA and reduced binding affinity to FcRIIB compared to the antibody having the wild-type IgG1 Fc sequence of SEQ ID NO: 1.


SEQUENCE LISTING (4 sequences)

SEQ ID NO: 1 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 4 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK